(19) United States
(12) Patent Application Publication
(10) Pub. No.: US 2022/0090149 A1
(43) Pub. Date: Mar. 24, 2022

(54) MICROBIAL STRAINS ENGINEERED FOR IMPROVED FRUCTOSE UTILIZATION

(71) Applicant: PTT GLOBAL CHEMICAL PUBLIC COMPANY LIMITED, Bangkok (TH)

(72) Inventors: Sudhanshu Vijay DOLE, Andover, MA (US); Joel Stewart SCHMID, Nashua, NH (US); R. Rogers YOCUM, Lexington, MA (US); Theron HERMANN, Arlington, MA (US); Russell Lizardo UDANI, Arlington, MA (US); Sean Joseph REGAN, Worcester, MA (US); Mark Andrew SHEFF, Westwood, MA (US); Michelle SPENCER, Woburn, MA (US); Ryan SILLERS, Westwood, MA (US); Phatthanon PRASITCHOKE, Bangkok (TH); Natthawut POOMSILA, Bangkok (TH)

(73) Assignee: PTT GLOBAL CHEMICAL PUBLIC COMPANY LIMITED, Bangkok (TH)

(21) Appl. No.: 17/416,885

(22) PCT Filed: Dec. 23, 2019
(86) PCT No.: PCT/IB2019/001363

§ 371 Date: Jun. 21, 2021

Related U.S. Application Data
(60) Provisional application No. 62/783,661, filed on Dec. 21, 2018.

(51) Int. Cl. C12P 7/56; C12N 9/12; C07K 14/395

(52) U.S. Cl. CPC C12P 7/56; C12N 9/1205; C07K 14/395; C12Y 207/01001; C12Y 207/01004

(57) ABSTRACT

The present invention discloses a genetically engineered Kluyveromyces sp. yeast strain that is capable of producing lactic acid from carbon source selected from glucose, fructose, sucrose or a mixture thereof wherein the genetically engineered yeast comprises at least one heterologous DNA cassette that confers production of a protein functioning as a fructose importer. The genetically engineered yeast strain according to this invention has an improvement of fructose utilization and use fructose as a faster rate than conventional strain, allowing for shorter fermentation times and improved economics.

### CROSS-REFERENCE TO RELATED APPLICATIONS

Not applicable

### STATEMENT REGARDING FEDERALLY SPONSORED RESEARCH OR DEVELOPMENT

Not applicable

### JOINT RESEARCH AGREEMENT

Not applicable

### REFERENCE TO SEQUENCE LISTING

The Sequence Listing is incorporated by reference herein.

### FIELD OF INVENTION

The invention relates to the field of genetic engineering of micro-organisms for chemical production. More specifically, the invention relates to the use of carbon source selected from glucose, fructose, sucrose or a mixture thereof for producing lactic acid using genetically modified microorganisms.

### BACKGROUND OF INVENTION

The current inventors and others have engineered Kluyveromyces marxianus yeast strains to produce D-lactic acid or L-lactic acid (for two representative examples, see US Provisional Patent application 62-631,541 and U.S. Pat. No. 7,534,597). In all of the prior art examples except for U.S. patent application 62/631,541, dextrose is used as the carbon source in biological fermentation. However, in Thailand and Southeast Asia and other warm or tropical locations such as Brazil, Central America, India, Southern US, etc., a preferred carbon source for biological fermentation is the disaccharide sucrose, derived from sugar cane juice. Similarly, in temperate regions where sugar beets are grown, sucrose can be a preferred carbon source for biological fermentation.

K. marxiamus, as well as many other yeasts, such as Saccharomyces cerevisiae, secretes an invertase enzyme into the fermentation medium or periplasmic space, the enzyme cleaves sucrose into two monosaccharides, D-glucose (D-glucose, also known as dextrose, shall be hereinafter referred to simply as glucose for brevity) and D-fructose (which shall be hereinafter referred to simply as fructose for brevity). The two monosaccharides are then imported into the yeast cells and metabolized.

Most microbes, including the two yeast species just mentioned, have a preference for metabolizing glucose when presented with a mixture of glucose and any other carbon source, such as fructose. This preference is generally accomplished by repressing or inhibiting the use of non-glucose carbon sources by any one of several different mechanisms that are given a variety of names, such as glucose inhibition, glucose repression, catabolite repression, carbon catabolite repression, and inducer exclusion. We noticed that in most of our fermentations that used one of our D-lactic acid or L-lactic acid producing strains and sucrose as the carbon source, there was usually some fructose remaining in the fermentation broth at the end of fermentation, typically around 48 hours (see FIG. 17). The fructose concentration typically varied from about 1 g/L to about 20 g/L, while the glucose concentration was typically below detection. This pattern proves that on average, the fructose is used more slowly than the glucose, since cleavage of sucrose gives an equimolar amount of glucose and fructose. The presence of residual fructose is undesirable, since it is a reducing sugar and can therefore react with amino compounds in “Maillard Reactions” and/or caramelization reactions that results in unwanted yellow- and brown-colored compounds that are difficult to remove in the downstream processing of the lactic acid. Furthermore, the residual fructose is a form of wasted carbon, since it is impractical to rescue it from the waste stream. If the fructose could be utilized at a faster rate, a higher yield of product from sucrose could be attained. Thus, improved fructose utilization would be useful for two good reasons, 1) unwanted byproducts would be reduced, and 2) a higher yield of the desired product would be obtained.

Having typically seen residual fructose in Kluyveromyces marxianus fermentation that produced D-lactic acid or L-lactic acid, we then showed that the same phenomenon occurs when Saccharomyces cerevisiae distillery strains are grown on sucrose or mixtures of glucose and fructose for ethanol production. At a time when glucose has been completely consumed, we found residual fructose.

For some commercial bacterial fermentations, sucrose is again a preferred carbon source, and a similar problem occurs, namely that residual fructose remains in the fermentation broth after the glucose is consumed (for example, see FIG. 1 in U.S. Pat. No. 9,845,513).

We shall use the phrase “fructose problem” or “the fructose problem” to mean a phenomenon in which a microbial strain consumes glucose at a faster rate than it consumes fructose, on average, or at any other time during growth or fermentation in a medium that contains both glucose and fructose at some stage during the growth or fermentation. The mixture of glucose and fructose can be present in the medium at the beginning of growth or fermentation, or said mixture can be generated by hydrolysis of sucrose during growth or fermentation. Thus, as a result of the above mentioned glucose repression of fructose utilization, when many microbial species are grown on sucrose as a carbon source, the resulting glucose is consumed more quickly than the resulting fructose, such that commercial fermentations, using sucrose or mixtures containing glucose and fructose, need to be run for a longer time than fermentations that use only glucose, in order for all of the sugar to be consumed and converted into the desired product, such as ethanol, one or more butanol isomer, D-lactic acid, L-lactic acid, succinic acid, malic, citric acid, a carotenoid, isoprene, a lipid, or any other chemical of commercial interest. The goal of this invention was to increase the rate of fructose utilization by any suitable or desirable microbial strain for fermentations from carbon source selected from glucose, fructose, sucrose or a mixture thereof.

### CURRENT STATE OF THE ART

The inventors are not aware of any engineering of commercial yeast strains for the purpose of improving fructose utilization. Ethanol fermentations in Brazil that use sugar cane juice and molasses and conventional yeast strains are simply run until all sugars have been consumed Commercial L-lactic acid and succinic acid fermentations at Cargill and BioAmber are believed to use an engineered Issatchenkia orientalis yeast that is based on glucose as the sole carbon source, since the parent strain does not use sucrose. Cargill has filed a US patent application that describes addition of an invertase gene to their I. orientalis succinate producer, but the resulting strain evidently has the “fructose problem” as defined above (see FIG. 1 of WO 2017/091610 A1). WO 2017/091610 A1 also discloses the concept of producing “lactic acid” by a yeast that produces an invertase. However, WO 2017091610 A1 does not distinguish between D-lactic acid and L-lactic and does not disclose how to engineer a yeast strain to economically produce D-lactic acid or L-lactic acid using sucrose or a mixture that includes glucose and fructose, or how to produce D-lactic acid or L-lactic acid by such a yeast in an economically attractive process, using sucrose or a mixture that includes glucose and fructose. Furthermore WO 2017/091610 A1 ignores the prior art that disclosed L-lactic acid production by engineered strains of Saccharomyces cerevisiae or Kluyveromyces lactis in which one or more genes encoding pyruvate decarboxylase have been deleted, and which natively secrete an invertase (U.S. Pat. No. 7,049,108 B2). Although U.S. Pat. No. 7,049,108 B2 discloses the concept of producing L-lactic acid or D-lactic acid, the strains and methods disclosed, once again, do now allow for an economically attractive process that can compete with current commercial processes, for example those that use a bacterium such as Bacillus coagulans as the production organism (Poudel, 2016 #124); Michelson, 2006 #123). An “economically attractive” process for producing an isomer of lactic acid is a process in which is performed by a yeast strain that is capable of producing D-lactic acid or L-lactic acid from sucrose and/or a mixture of glucose and fructose at titer of at least 110 g/L, at a final pH of less than 3.7, with a yield on sugar of at least 0.75 g/g, in 48 hours or less.

US 2011/0256598 proposed a fucose:H+ symporter from Escherichia coli to be used to increase import of fructose in microbes, but the inventors did not demonstrate the use of this importer in yeast, so it is not clear that it would function in yeast.

(Pina, 2004 #121) describe the cloning of a gene from Zygosaccharomyces bailii encoding a fructose transporter, which was designated FFZ1 (fructose facilitator Zygosaccharomyces). The transporter was shown to function in Saccharomyces cerevisiae. However, the authors did not mention growth on sucrose or mixtures of glucose and fructose, or improved fructose utilization in the presence of glucose.

(Zhou, 2017 #1) also demonstrated the functioning of fructose importers encoded by “fziI” or “fsy1” from Candida magnoliae in S. cerevisiae, and proposed their use for help in consuming fructose in strains designed to produce melibiose from raffinose, but again, the authors did not mention growth on sucrose or mixtures of glucose and fructose, or improved fructose utilization in the presence of glucose.

As such there is still a need for microbial strains that have improved fructose utilization when sucrose or a mixture of glucose and fructose is present as significant carbon sources in a fermentation medium.

### SUMMARY OF INVENTION

The goal of this invention, namely to improve fructose utilization in the presence of glucose by a wide variety of microbial strains, was achieved by introducing a gene cassette designed to express the gene FZ1 from Zygosaccharomyces rouxii (which we shall refer to hereinafter as ZrFFZ1), which is cloned from a so-called fructophilic yeast, which is a yeast that naturally consumes fructose at a rate faster that it consumes glucose. How to achieve a useful level of expression of an FFZ1 gene in a desired heterologous organism, as measured by an increased rate of fructose utilization in the presence of glucose, was not obvious, as the previously published FFZ1 gene from Z. bailii (which will shall refer to as ZbFFZ1) did not function measurably in K. marxiamus in a construction parallel to that comprising the ZrFFZ1 gene.

In the invention disclosed herein, a surprising discovery was made, namely that addition of an expressed FFZ1 gene to non-fructophilic yeasts increased their rate of fructose utilization relative to glucose utilization, despite the fact that the parent yeast strains already had an inherent ability to utilize fructose. This improvement is useful for more complete utilization of fructose in shorter fermentation times.

The present invention discloses a genetically engineered Kluyveromyces sp. yeast strain that is capable of producing lactic acid from carbon source selected from glucose, fructose, sucrose or a mixture thereof wherein the genetically engineered yeast comprises at least one heterologous DNA cassette that confers production of a protein functioning as a fructose importer. The genetically engineered yeast strain according to this invention has an improvement of fructose utilization and use fructose as a faster rate than conventional strain and use fructose at a faster rate than conventional strain, allowing for shorter fermentation times and improved economics.

### BRIEF DESCRIPTION OF DRAWINGS

FIG. 1. Structure of DNA cassette for deletion of NEJ1

FIG. 2. Structure of DNA cassette for integration and expression of FFZ1 at the ADH2 Locus on Chromosome 4.

FIGS. 3A AND 3B. Ratios of fructose used to glucose used in BioLector fermentations by strains derived from MYR2785 that contain an integrated cassette designed to express either ZbFFZ1 or ZrFFZ1 in 12% sucrose medium and 6% glucose and 6% fructose medium, respectively.

FIG. 4. Structure of pMS155, which contains a cassette designed to replace any of the three EcldhA expression cassettes in SD1774 with a PaldhL expression cassette, to convert D-lactate producing strains of the invention to L-lactate producing strains.

FIG. 5. Structure of cassette JSS89 for expression of ZrFFZ1 to be integrated by Insertion in the Middle of the KMADH6 Open Reading Frame.

FIG. 6. Structure of cassette JSS90 designed for expression of ZrFFZ1 to be integrated at KmADH6 with simultaneous deletion of the KmADH6 open reading frame.

FIG. 7. [Fructose Used]/[Glucose Used] by strains containing either the JSS89 or JSS90 cassette in a BioLector Fermentation with a 12% Sucrose medium at 75 hours.

FIG. 8. Ratio of [fructose used]/[glucose used] by L-lactate producing strains containing an integrated ZrFFZ1 expression cassette, grown in shake flasks buffered with calcium carbonate for 96 hours

FIG. 9. Consumption of sugars by Ethanol Red grown under microaerobic conditions in a minimal medium containing 12% sucrose.

FIG. 10. Consumption of sugars by Ethanol Red grown under microaerobic conditions in a minimal medium containing 6% fructose plus 6% glucose.

FIG. 11. Structure of pRY789, a plasmid that contains a cassette for integrating ZrFFZ1 at the HO locus of Saccharomyces cerevisiae.

FIG. 12. Fructose and glucose utilization by Ethanol Red without or with the ZrFFZ1 expression cassette from pRY789 installed, grown under microaerobic conditions in a minimal medium containing 6% fructose and 6% glucose.

FIG. 13. Comparison of L-lactic acid titers of strains JSS1397 and KMS1017 in 7-liter fermenters.

FIG. 14. Comparison of fructose concentrations in 7-liter fermentations of strains JSS1397 and KMS1017.

FIG. 15. Ratio of rate of glucose utilization to rate of fructose utilization by strains JSS1397 and KMS1017 in 7-liter fermenters.

FIG. 16. L-Lactic acid titers vs. pH in 7-liter fermentors with solubility limit shown as solid line.

FIG. 17. Residual fructose concentrations in pH-controlled 7-liter fermentations of a D-lactic acid producing yeast strain that contains a ZrFFZ1 cassette (strain SD1755), two ZrFFZ1 cassettes (MYR2879) and does not contain such a cassette (strain MYR2785) with an initial batch feed of 180 g/L sucrose.

FIG. 18. Structure of pBc-ldhL-OP2-int, which contains a cassette designed to replace any of the three EcldhA expression cassettes in MYR2787 with a BcldhL expression cassette, to convert D-lactate producing strains of the invention to L-lactate producing strains.

FIG. 19. Residual fructose concentrations in pH-controlled 5-liter fermentations of a L-lactic acid producing yeast strain that contains a ZrFFZ1 cassette (strain JSS1397) and does not contain a ZrFFZ1 cassette (strain MYR2893), both with an initial batch feed of 216.7 g % L canejuice.

FIG. 20. Residual fructose concentrations in pH-controlled 5-liter fermentations of a L-lactic acid producing yeast strain that contains two ZrFFZ1 cassettes (strain JSS1397) and two ZrFFZ1 cassettes and KmRAG5 (MYR3059) with an initial batch feed of 150 g/L sucrose.

FIG. 21. L-Lactic acid concentrations in pH-controlled 5-liter fermentations of a L-lactic acid producing yeast strain that contains two ZrFFZ1 cassettes (strain JSS1397) and two ZrFFZ1 cassettes and KmRAG5 (strain MYR3059) with an initial batch feed of 150 g/L sucrose.

### DETAILED DESCRIPTION OF INVENTION

A relatively small number of yeast species prefer to utilize fructose over glucose when presented with a mixture of the two. Such yeasts are called “Fructophiles”, are said to be “Fructophilic”, or are said to exhibit “Fructophily”. Examples of fructophilic yeasts are members of the genus Zygosaccharomyces, such as Z. rouxii and Z. bailii (Leandro, 2014 #6), members of the Wickerhamiella/Starmerella or W/S clade (Goncalves, 2018 #4), and some yeasts in the genus Candida, such as Candida magnoliae (Zhou, 2017 #1)).

An obligate feature of many, if not all, fructophilic yeasts is the FFZ1 gene, or a homolog thereof. The set of FFZ1 genes and their homologs encode a high capacity but low affinity uniporter (also known as a “facilitated diffuser” or simply a “facilitator”, that is specific for fructose. We shall refer to such fructose importer proteins as Ffz1. Ffz1 proteins are membrane proteins that function to facilitate fructose diffusion down a concentration gradient into the cell, after which the fructose can be metabolized. Wild type non-fructophilic yeasts, such as K. marxianus, S. cerevisiae, Issatchenkia orientalis, and Pichia pastoris, have the ability to metabolize fructose by virtue of having hexose importers and metabolic pathways for using fructose. However, as the name implies, the typical hexose transporters, which are encoded by genes such as HXTn from S. cerevisiae, where n is an integer from 1 to 17, and their homologs and analogs, also transport glucose, and because of a large number of factors, including regulation of gene expression, protein activity, and differential affinity, most or all the HTXn encoded hexose transporters favor glucose over fructose. Because the Hxtn importer proteins can help to diffuse both glucose and fructose into cells, both sugars are ultimately consumed in fermentations based largely on S. cerevisiae where both fructose and glucose are present, such as cane sugar (and or molasses) tin ethanol fermentations. The same is the case for fermentations based largely on K. marxianus and K. lactis yeasts, which also use HTXn homologs for hexose import. However, in such fermentations, the rate of glucose consumption is typically higher than the rate of fructose consumption. In any case, for some industrial fermentations, such as those which produce products such as fuel ethanol, one or more butanol isomer, D-lactic acid, L-lactic acid, succinic acid, malic, citric acid, a carotenoid, isoprene, or a lipid of commercial interest, there is still a need to shorten fermentation times by increasing the rate at which fructose is consumed, especially when glucose is also present in the medium, by yeast species and strains that are not naturally fructophilic.

Various non-limiting embodiments of the disclosure will now be described herein and illustrated in the accompanying drawings. A person having ordinary skill in the art will understand that the features, structures, components, or characteristics described or illustrated in connection with one non-limiting embodiment may be combined with the features, structures, components, or characteristics of one or more other non-limiting embodiments. Such combinations are intended to be included within the scope of the disclosure. A person having ordinary skill in the art will also understand that that the features, structures, components, or characteristics described or illustrated in connection with one or more non-limiting embodiments can be modified or varied without departing from the scope and spirit of the invention.

To facilitate understanding of the invention, a description of nomenclature is provided below.

In regards to nomenclature, a bacterial gene or coding region is usually named with lower case letters in italics, for example “ldhA” from E. coli, while the enzyme or protein encoded by the gene can be named with the same letters, but with the first letter in upper case and without italics, for example “LdhA”. A yeast gene or coding region is usually named with upper case letters in italics, for example “PDC1”, while the enzyme or protein encoded by the gene can be named with the same letters, but with the first letter in upper case and without italics, for example “Pdc1” or “Pdc1p”, the latter of which is an example of a convention used in yeast for designating an enzyme or protein. The “p” is an abbreviation for the protein encoded by the designated gene. The enzyme or protein can also be referred to by a more descriptive name, for example, D-lactate dehydrogenase or pyruvate decarboxylase, referring respectively to the two above examples. A gene or coding region that encodes one example of an enzyme that has a particular catalytic activity can have several different names because of historically different origins, functionally redundant genes, genes regulated differently, or because the genes come from different species. For example, a gene that encodes glycerol-3-phosphate dehydrogenase can be named GPD1, GDP2, or DARI, as well as other names. To specify the organism from which a particular gene was derived, the gene name can be preceded by two letters indicating the genus and species. For example, the KmURA3 gene is derived from Kluyveromyces marxianus, the ScURA3 gene is derived from Saccharomyces cerevisiae, the EcldhA gene is derived from E. coli, the PaldhL gene is derived from Pediococcus acidilactici, and the BcldhL is derived from Bacillus coagulans. For yeast strains that contain a mutation in particular gene, or have a mutant phenotype, the gene or strain is designated by lower case italicized letters, for example ura3 or ura3. for a strain that lacks a functional URA3 gene.

Note that all isomers of lactic acid and any lactic acid analog can exist in solid, liquid, or solution form as a protonated acid (also known as a free acid) or as an ionized salt. In aqueous solution, both protonated and ionic forms co-exist in an equilibrium. Since it would be cumbersome to refer to all forms of such compounds at every instance, any mention of either an acid form or a salt form (for example D-lactic acid (D-LAC), D-lactate, L-lactic acid (L-LAC), L-lactate, or D,L-beta-chlorolactate) includes all forms or mixtures thereof.

To facilitate understanding of the invention, a number of terms are defined below, and others are found elsewhere in the specification.

“Yeast” means any fungal organism that is capable of growing in a single cell state under some conditions. Some yeast strains can also grow in a hyphal state or pseudohyphal (i.e., short hyphae) state under some conditions, such as under starvation. In particular, yeast includes, but is not limited to, organisms in the genera Saccharomyces, Kluveromyces, Issatchenkia, Pichia, Hansenula, Candida, Yarrowia, Zygosaccharomyces, Schizosaccharomyces, and Lachancea.

“Cassette” or “expression cassette” means a deoxyribose nucleic acid (DNA) sequence that is capable of encoding, producing, or overproducing, or alternatively, eliminating or reducing the activity of, one or more desired proteins or enzymes when installed in a host organism. A cassette for producing a protein or enzyme typically comprises at least one promoter, at least one protein coding sequence (also known as an “open reading frame” or “ORF”), and optionally at least one transcription terminator. If a gene to be expressed is heterologous or exogenous, the promoter and terminator are usually derived from two different genes or from a heterologous gene, in order to prevent double recombination with the native gene from which the promoter or terminator was derived. A cassette can optionally and preferably contain one or two flanking sequence(s) on either or both ends that is/are homologous to a DNA sequence in a host organism (a “target” sequence), such that the cassette can undergo homologous recombination with the host organism, either with a chromosome or a plasmid, at the target sequence, resulting in integration of the cassette into said chromosome or plasmid at the target sequence. If only one end of a cassette contains a flanking homology, then the cassette in a circular format can integrate by single recombination at the flanking sequence. If both ends of a cassette contain flanking homologies, then the cassette in a linear or circular format can integrate by double recombination with the surrounding flanks. A cassette can be constructed by genetic engineering, where for example a coding sequence is expressed from a non-native promoter, or it can use the naturally associated promoter. A cassette can be built into a plasmid, which can be circular, or it can be a linear DNA created by polymerase chain reaction (PCR), primer extension PCR, or by in vivo or in vitro homologous recombination between ends of DNA fragments, each of which is a subset of the desired final cassette, where each subset fragment has an overlapping homology at either or both ends, designed to result in joining of adjacent fragments by homologous recombination either in vitro or in in vivo. A cassette can be designed to include a selectable marker gene or DNA sequence that upon integration is surrounded by a direct repeat sequence of about 30 base pairs or more (the same sequence, in the same orientation present at both ends of the integrated selectable gene), such that the selectable marker can be deleted by homologous recombination between the direct repeats (also known as “looping out”), after the initial cassette containing the selectable marker has been integrated into a chromosome or plasmid. Useful selectable marker genes include, but are not limited to, antibiotic G418 resistance (kan or kanR), hygromycin resistance (hyg or hygR), zeocin resistance (zeo or zeoR), naturicin resistance (nat or natR), and biosynthetic genes such as URA3, TRP1, TRP5, LEU2, and HIS3. For the biosynthetic genes to be used as a selectable marker, the host strain must, of course, contain a mutation in the corresponding gene, preferably a non-reverting null mutation. For example, if URA3 is used as the selectable marker gene, then the strain to be transformed must be phenotypically ura3.. For the antibiotic resistance genes, the resistance gene usually requires a promoter that functions well enough in the host microbial strain to enable selection. Although a gene that is desired to be expressed can be installed in a host strain in the form of a cassette, a gene, for example a coding sequence from start codon to stop codon can be integrated into a host chromosome or plasmid without a promoter or terminator such that the incoming coding sequence precisely or approximately replaces the coding sequence of a gene native to the host strain, such that after integration, the incoming coding region is expressed from the remaining promoter of the host coding sequence that was replaced by the incoming coding sequence.

In some of the examples described herein, cassettes were assembled in vivo by transforming a yeast strain with a mixture of roughly equimolar concentrations of two or more linear DNA fragments that are joined together inside the cell by homologous recombination using “overlapping homology”, in which relatively short DNA sequences (about 20 to 50 base pairs, at each end of a subset fragment are identical to the sequence of an adjacent subset fragment in the final assembled cassette, or to the chromosomal target sequence for the 5′ and 3′ ends of the final assembled cassette. Many yeast strains, including K. marxianus and S. cerevisiae have the ability to assemble the multiple subset fragments into the final cassette and integrate the assembled cassette into a chromosomal target, all by homologous recombination between the “overlapping homologies”.

“D-lactate dehydrogenase” means any enzyme that catalyzes the formation of D-lactate from pyruvate. “L-lactate dehydrogenase” means any enzyme that catalyzes the formation of L-lactate from pyruvate. The necessary reducing equivalent for either of these reactions can be supplied by NADH, NADPH, or any other reducing equivalent donor.

“Gibson method” means a method for joining in vitro together two or more linear DNA fragments that have short (about 15-40 base pairs) overlapping homology at their ends. This method can be used to construct plasmids from synthetic linear DNA fragments, PCR fragments, or fragments generated by restriction enzymes. Kits can be purchased to perform the Gibson method, for example the NEBuilder HiFi DNA Assembly Cloning Kit (New England BioLabs, Ipswitch, Mass., USA), and used as instructed by the manufacturer.

“Transformant” means a cell or strain that results from installation of a desired DNA sequence, either linear or circular, and either autonomously replicating or not, into a host or parent strain.

“Titer” means the concentration of a compound in a fermentation broth, usually expressed as grams per liter (g/L) or as % weight per volume (%). Titer is determined by any suitable analytical method, such as quantitative analytical chromatography, for example high pressure liquid chromatography (HPLC) or gas chromatography (GC), with a standard curve made from external standards, and optionally with internal standards.

“Yield” means the grams of product per gram of carbon source used during fermentation. This is typically calculated based on titer, final liquid volume, and amount of carbon source supplied, with the final volume corrected for volumes sampled, fed, and/or evaporated. It is usually expressed as grams per gram (g/g) or as a % weight per weight (%).

“Time” means the time elapsed from inoculation to sampling or harvesting in a fermentation, typically measured in hours.

“Specific productivity” means the rate of product formation in grams of product produced in given volume of fermentation broth in a given period of time, typically expressed in grams per liter-hours (gL/hr). The “average specific productivity” means the specific productivity where the period of time is the entire fermentation from inoculation to sampling or harvest. The average specific productivity is lower than the specific productivity from the middle of a fermentation, since specific productivity is lower than average during the early growth period and during the later stages. Average specific productivity can be calculated by dividing final titer by the number of hours at harvest. Note that some published specific productivities are clearly not average specific productivities, although the period of measurement is not explicitly given (see Table 1 for some examples).

“pKa” means the pH at which an acid in solution is half in the conjugate base state, which is typically an ionic or salt form. The pKa for L-LAC and D-LAC is published to be from 3.78 to 3.86, although the exact pKa can vary slightly with temperature, concentration, and concentration of other solutes. For lactic acid, the conjugate base state is the lactate ion, so the pKa is the pH where the concentration of the lactate ion equals the concentration of the protonated or “free acid” state. The pKa can be measured by the well-known method of performing an acid-base titration and taking the midpoint of the titration curve. One skilled in the art will know that in aqueous solution, both D-lactic acid and L-lactic acid exist to some extent in the two forms, the protonated acid form and the ionized salt (i.e., conjugate base) form. As such, depending on context, the terms “D-lactate”, “D-lactic acid”, and “D-LAC” can mean either form, or a mixture of the two forms. In particular, when discussing titers and yields, the sum of both forms is meant to be included, but it is expressed in terms of the free acid, in other words, titer and yield is expressed as if any salt form that is present is converted to the free acid form.

“Heterologous” means a gene or protein that is not naturally or natively found in an organism, but which can be introduced into an organism by genetic engineering, such as by transformation, mating, or transduction. A heterologous gene can be integrated (i.e., inserted or installed) into a chromosome, or contained on a plasmid. The term “exogenous” means a gene or protein that has been introduced into, or altered, in an organism for the purpose of increasing, decreasing, or eliminating an activity, by genetic engineering, such as by transformation, mating, transduction, or mutagenesis. An exogenous gene or protein can be heterologous, or it can be a gene or protein that is native to the host organism, but altered by one or more methods, for example, mutation, deletion, change of promoter, change of terminator, duplication, or insertion of one or more additional copies in the chromosome or in a plasmid. Thus, for example, if a second copy of a DNA sequence is inserted at a site in the chromosome that is distinct from the native site, the second copy would be exogenous.

“Plasmid” means a circular or linear DNA molecule that is substantially smaller than a chromosome, is separate from the chromosome or chromosomes of a microorganism, and replicates separately from the chromosome or chromosomes. A plasmid can be present in about one copy per cell or in more than one copy per cell. Maintenance of a plasmid within a microbial cell usually requires growth in a medium that selects for presence of the plasmid, for example using an antibiotic resistance gene, or complementation of a chromosomal auxotrophy. However, some plasmids require no selective pressure for stable maintenance, for example the 2 micron circle plasmid in many Saccharomyces strains.

“Chromosome” or “chromosomal DNA” means a linear or circular DNA molecule that is substantially larger than a plasmid and usually does not require any antibiotic or nutritional selection. In the invention, a yeast artificial chromosome (YAC) can be used as a vector for installing heterologous and/or exogenous genes, but it would require selective pressure for maintenance.

“Overexpression” means causing the enzyme or protein encoded by a gene or coding region to be produced in a host microorganism at a level that is higher than the level found in the wild type version of the host microorganism under the same or similar growth conditions. This can be accomplished by, for example, one or more of the following methods: installing a stronger promoter, installing a stronger ribosome binding site, installing a terminator or a stronger terminator, improving the choice of codons at one or more sites in the coding region, improving the mRNA stability, or increasing the copy number of the gene either by introducing multiple copies in the chromosome or placing the cassette on a multicopy plasmid. An enzyme or protein produced from a gene that is overexpressed is said to be “overproduced.” A gene that is being overexpressed or a protein that is being overproduced can be one that is native to a host microorganism, or it can be one that has been transplanted by genetic engineering methods from a different organism into a host microorganism, in which case the enzyme or protein and the gene or coding region that encodes the enzyme or protein is called “foreign” or “heterologous.” Foreign or heterologous genes and proteins are by definition overexpressed and overproduced, since they are not present in the unengineered host organism.

“Homolog” means a second gene, DNA sequence, or protein sequence that is related by sequence homology to a different first gene, DNA sequence, or protein, wherein said second sequence has at least 25% sequence identity when comparing protein sequences or comparing the protein sequence derived from gene sequences, or at least 50% identity when comparing DNA sequences with said first gene, DNA sequence, or protein sequence, as determined by the Basic Local Alignment Search Tool (BLAST) computer program for sequence comparison (Altschul, 1990 #332; Altschul, 1997 #334), and allowing for deletions and insertions. An example of a homolog of the K. marxianus PDC1 gene would be the PDC1 gene from S. cerevisiae. A “functional homolog” is a second DNA or protein sequence that is a homolog and has been, or can be, shown to a have a function identical to, or similar to, said first DNA or protein sequence.

“Analog” means a gene, DNA sequence, or protein that performs a similar biological function to that of another gene, DNA sequence, or protein, but where there is less than 25% sequence identity (when comparing protein sequences or comparing the protein sequence derived from gene sequences) with said another gene, DNA sequence, or protein, as determined by the BLAST computer program for sequence comparison (Altschul, 1990 #26; Altschul, 1997 #17), and allowing for deletions and insertions. An example of an analog of the K. marxianus Gpd1 protein would be the K. marxianus Gut2 protein, since both proteins are enzymes that catalyze the same reaction, but there is no significant sequence homology between the two enzymes or their respective genes. A person having ordinary skill in the art will know that many enzymes and proteins that have a particular biological function (in the immediately above example, glycerol-3-phosphate dehydrogenase), can be found in many different organisms, either as homologs or analogs, and since members of such families of enzymes or proteins share the same function, although they may be slightly or substantially different in structure. Different members of the same family can in many cases be used to perform the same biological function using current methods of genetic engineering. Thus, for example, a gene that encodes D-lactate dehydrogenase could be obtained from any of many different organisms.

“Mutation” means any change from a native or parent DNA sequence, for example, an inversion, a duplication, an insertion of one or more base pairs, a deletion of one or more base pairs, a point mutation leading to a base change that creates a premature stop codon, or a missense mutation that changes the amino acid encoded at that position. “Null mutation” means a mutation that effectively eliminates the function of a gene. A complete deletion of a coding region would be a null mutation, but single base changes can also result in a null mutation. “Mutant”, “mutated strain”, “mutated yeast strain”, or a strain “that has been mutated” means a strain that comprises one or more mutations when compared to a native, wild type, parent or precursor strain.

The phrase “a mutation that eliminates or reduces the function of” means any mutation that lowers any assayable parameter or output, of a gene, protein, or enzyme, such as mRNA level, protein concentration, or specific enzyme activity of a strain, when said assayable parameter or output is measured and compared to that of the unmutated parent strain. Such a mutation is preferably a deletion mutation, but it can be any type of mutation that accomplishes a desired elimination or reduction of function.

“Strong constitutive promoter” means a DNA sequence that typically lies upstream (to the 5′ side of a gene when depicted in the conventional 5′ to 3′ orientation), of a DNA sequence or a gene that is transcribed by an RNA polymerase, and that causes said DNA sequence or gene to be expressed by transcription by an RNA polymerase at a level that is easily detected directly or indirectly by any appropriate assay procedure. Examples of appropriate assay procedures include quantitative reverse transcriptase plus PCR, enzyme assay of an encoded enzyme, Coomassie Blue-stained protein gel, or measurable production of a metabolite that is produced indirectly as a result of said transcription, and such measurable transcription occurring regardless of the presence or absence of a protein that specifically regulates the level of transcription, a metabolite, or an inducer chemical. By using well-known methods, a strong constitutive promoter can be used to replace a native promoter (a promoter that is otherwise naturally existing upstream from a DNA sequence or gene), resulting in an expression cassette that can be placed either in a plasmid or chromosome and that provides a level of expression of a desired DNA sequence or gene at a level that is higher than the level from the native promoter. A strong constitutive promoter can be specific for a species or genus, but often a strong constitutive promoter from a yeast can function well in a distantly related yeast. For example, the TEF1 (translation elongation factor 1) promoter from Ashbya gossypii functions well in many other yeast genera, including K. marxianus.

“Microaerobic” or “microarobic fermentation conditions” means that the supply of air to a fermenter is less than 0.1 volume of air per volume of liquid broth per minute (vvm).

“Chemically defined medium”, “minimal medium”, or “mineral medium” means any fermentation medium that is comprised of purified chemicals such as mineral salts (for example sodium, potassium, ammonium, magnesium, calcium, phosphate, sulfate, chloride, etc.) which provide necessary element such as nitrogen, sulfur, magnesium, phosphorus (and sometimes calcium and chloride), vitamins (when necessary or stimulatory for the microbe to grow), one or more pure carbon sources, such as a pure sugar, glycerol, ethanol, etc., trace metals as necessary or stimulatory for the microbe to grow (such as iron, manganese, copper, zinc, molybdenum, nickel, boron and cobalt), and optionally an osmotic protectant such as glycine betaine, also known as betaine. Except for the optional osmoprotectant and vitamin(s), such media do not contain significant amounts of any nutrient or mix of more than one nutrient that is not essential for the growth of the microbe being fermented. Such media do not contain any significant amount of rich or complex nutrient mixtures such as yeast extract, peptone, protein hydrolysate, molasses, broth, plant extract, animal extract, microbe extract, whey, Jerusalem artichoke powder, and the like. For producing a commodity chemical by fermentation where purification of the desired chemical by simple distillation is a not an economically attractive option, a minimal medium is preferred over a rich medium because a minimal medium is usually less expensive, and the fermentation broth at the end of fermentation usually contains lower concentrations of unwanted contaminating chemicals that need to be purified away from the desired chemical.

“Fermentation production medium” means the medium used in the last tank, vessel, or fermentor, in a series comprising one or more tanks, vessels, or fermentors, in a process wherein a microbe is grown to produce a desired product (for example D-LAC or L-LAC). For production of a commodity chemical by fermentation such as D-LAC or L-LAC, where extensive purification is necessary or desired, a fermentation production medium that is a minimal medium is preferred over a rich medium because a minimal medium is often less expensive, and the fermentation broth at the end of fermentation usually contains lower concentrations of unwanted contaminating chemicals that need to be purified away from the desired chemical. Although it is generally preferred to minimize the concentration of rich nutrients in such a fermentation, in some cases it is advantageous for the overall process to grow an inoculum culture in a medium that is different from the fermentation production medium, for example to grow a relatively small volume (usually 10% or less of the fermentation production medium volume) of inoculum culture grown in a medium that contains one or more rich ingredients. If the inoculum culture is small relative to the production culture, the rich components of the inoculum culture can be diluted into the fermentation production medium to the point where they do not substantially interfere with purification of the desired product. A fermentation production medium must contain a carbon source, which is typically a sugar, glycerol, fat, fatty acid, carbon dioxide, methane, alcohol, or organic acid. In some geographic locations, for example in the Midwestern United States, D-glucose (dextrose) is relatively inexpensive and therefore is useful as a carbon source. Most prior art publications on lactic acid production by a yeast use dextrose as the carbon source. However, in some geographic locations, such as Brazil and much of Southeast Asia, sucrose is less expensive than dextrose, so sucrose is a preferred carbon source in those regions.

“Final pH” means the pH of a fermentation broth at the end of a fermentation when the fermentation is considered complete, fermentation is stopped, and the broth is harvested. Although it is preferred that the final pH of a lactic acid fermentation be below the pKa of lactic acid, it is also preferred that the pH during fermentation be controlled by addition of a “base” (an alkaline substance), to prevent the pH from falling too quickly or ending too low. The “base” can be in a solution, suspension, slurry, or solid form. The “base” can be a hydroxide, oxide, carbonate, or bicarbonate salt of sodium, ammonium, potassium, magnesium, or calcium. For production of lactic acid, a preferred base is a slurry of calcium hydroxide or powdered calcium hydroxide, which leads to the formation of some calcium lactate mixed with the protonated acid form in the fermentation broth. The resulting fermentation broth at the end of fermentation can be treated with sulfuric acid, which causes precipitation of calcium sulfate (gypsum), which aids in the removal of calcium, to increase the proportion of the lactic acid that is present in the protonated form. The feeding of the base to control pH can be done manually or by an automatically controlled pump or auger, as called for by pH measurements, which can be obtained manually or by continuous monitoring through a pH probe immersed in the fermentation vessel.

To facilitate understanding of the invention, various genes are listed in Table 1.

General Methods and Materials. Unless otherwise specified, recombinant DNA and genetic engineering was carried out with methods and materials well known in the art. Plasmids and linear DNA cassettes were assembled using the “Gibson method” with the NEBuilder HiFi DNA Assembly Cloning Kit (New England Biolabs) according to the manufacturers protocol, or by in vivo homologous recombination as described above. For in vivo assembly of a cassette, the subset DNA fragments are supplied in a roughly equimolar mixture, ideally containing a total of at least 500 ng of DNA. Thus, for example, in vivo assembly of cassette from two subset pieces that are 1000 bp (base pairs) and 2000 bp in length should use at least 166 and 333 ng of the two fragments, respectively. The larger the number of fragments to be assembled in vivo, the more DNA is required to increase the probability of obtaining a successful assembly. In one extreme case (see Example 5), six fragments were assembled and integrated in vivo in one transformation. The total amount of DNA for the six fragments was about 5 μg, about 40 transformants were obtained, and about 10 out of those 40 had the desired integrated structure. All of the component or “subset” DNA fragments used for assembling linear cassettes or plasmids, including plasmid backbones where appropriate, were generated by one of three methods, as appropriate: 1) by restriction enzyme cutting from precursor DNA sequences according to the suppliers instructions, 2) by PCR (Polymerase Chain Reaction) using Phusion High Fidelity PCR Master Mix (New England Biolabs) according to the manufacturers protocol, or 3) commercial synthesis of gBlocks by Integrated DNA Technologies, Inc.

Correct constructions were identified and/or confirmed by either diagnostic restriction enzyme digestion and agarose gel electrophoresis (for example in the case of plasmids generated by Qiagen Miniprep Kits), or by appropriate diagnostic PCR, for example in which the PCR product crosses the junction(s) of two or more adjacent precursor fragments to confirm correct joining, and agarose gel electrophoresis, using either Phusion High Fidelity PCR Master Mix (New England Biolabs), or Phire Plant PCR Master Mix Kit (ThermoScientific), according to the suppliers protocol. Correct structure of cassettes integrated into a yeast chromosome were identified by appropriate diagnostic PCR, for example in which a first PCR primer reads outward from within the cassette to be integrated, and a second primer reads toward the integration junction and the first PCR primer, from an adjacent chromosomal sequence that flanks the targeted integration site, but is not contained in, the cassette to be integrated. Diagnostic PCR to identify correct DNA structures can be performed on whole cells (for example either E. coli or yeast transformants containing a plasmid or an integrated linear DNA cassette). Approximately one to two microliters of cell volume is picked from a colony on a petri plate with a toothpick or micropipette tip and the cells are suspended in 20 microliters of sterile water or “Dilution Buffer” from the Phire Plant PCR Master Mix Kit (ThermoScientific). Then one microliter of such a cell suspension is used as the template DNA in a 20 or 25 microliter (total volume) PCR reaction for 25 to 40 cycles. Alternatively, an approximately equivalent number of cells can be obtained for use as the template DNA by pelleting approximately 100 microliters of a saturated liquid culture in a microfuge, removing the supernatant, and resuspending the cell pellet in 20 microliters of sterile water or Dilution Buffer.

In some cases, for example where a diagnostic PCR indicates a correct structure, but other evidence indicates lack of expected function, all or part of a cassette, or a PCR product amplified from a plasmid-borne or chromosomally integrated cassette, is sequenced to confirm or disprove the desired or expected DNA sequence. Many commercial companies perform DNA sequencing services, for example GeneWiz, Cambridge, Mass., USA.

To delete a DNA sequence or to integrate an expression cassette, the method that we generally used to assemble the cassette on a plasmid that can replicate in E. coli, or to assemble the cassette in vivo in the target yeast strain by co-transforming two or more subsections of the cassette, with adjacent subsections designed to overlap by 40 to 60 base pairs at the ends to be joined, as well as 40 to 60 base pairs at the ends of the assembled cassette that are homologous with the chromosomal target sequence. All of the cassettes described herein for integration in a K. marxianus chromosome were designed to express a yeast URA3 gene (typically the ScURA3 gene or the native KmUR43 gene) and the recipient host organism has a non-reverting ura3− phenotype, typically by virtue of a deletion at the native KmURA3 locus. In order to be able to re-use the URA3+ selection in subsequent engineering steps, in each cassette, the URA3 gene is surrounded by a direct repeat DNA sequences that allows deletion of the URA3 gene from the cassette after it has been integrated, by homologous recombination between said directly repeated DNA sequences, in a second step by selecting against the URA3 gene on minimal medium containing 5-fluoroorotic acid (see US application U.S. patent application 62/631,541 for details). Thus an integration cassette that is designed to insert between two particular base pairs at a chromosomal target site, when assembled in a plasmid or directly into a yeast chromosome, has the general structure, in order, the following subsections or precursor DNA fragments: 1) a sequence of 40 or more base pairs that is homologous to the target chromosomal sequence that is just upstream from the desired integration target site, labeled in the Figures as “Up”, 2) a DNA sequence that is desired to be integrated, for example a promoter-ORF-terminator combination, 3) a sequence “DR” (for Direct Repeat) of 40 or more base pairs that is not homologous to any sequence near the target chromosomal sequence, 4) a selectable gene such as the URA3 gene, 5) a second copy of the DR sequence of fragment 3, and 6) a sequence of 40 or more base pairs that is homologous to the target chromosomal sequence that is just downstream from the desired integration target site, labeled in the Figures as “Down”. In this case, the cassette integrates by double homologous recombination between “Up” and “Down”. Upon counterselection of the selectable gene, homologous recombination between the two copies of “DR” results in the looping out of the selectable gene, leaving the desired sequence precisely inserted between two specific base pairs at the chromosomal target.

In an alternative cassette design, in which it is desired to delete a DNA sequence at the chromosomal target site, the cassette, when assembled, will have the general structure, in order, the following subsections or precursor DNA fragments: 1) a sequence of 40 or more base pairs that is homologous to the target chromosomal sequence that is just upstream from the desired integration target site, labeled in the Figures as “Up”, 2) a DNA sequence that is desired to be integrated, for example a promoter-ORF-terminator combination, 3) a sequence “Down” of 40 or more base pairs that is homologous to the target chromosomal sequence just downstream of the desired deletion endpoint, 4) a selectable gene such as the URA3 gene, 5) a DNA sequence “Middle” of at least 40 base pairs that is homologous to at least a portion of the chromosomal target sequence that is desired to be deleted. In the case where a clean deletion desired and no DNA is desired to be inserted, then the second fragment is this design is omitted. Upon transformation and selection, the assembled cassette integrates into the chromosomal target site by homologous double recombination between the “Up” sequence and the “Middle” sequence. Correct integration of the entire assembled cassette is verified by diagnostic PCR In a second step the selectable gene is “looped out” by counterselection and homologous recombination between the “Down” sequence internal to the cassette, and the sequence that is homologous to “Down” in the chromosome that is logically present downstream from the integrated cassette.

The following examples are provided to further explain the invention but are not intended to limit the scope of the invention.

### Example 1. Method for DNA Transformation of Kluyveromyces Marxianus Strain SD 98 and its Derivatives

The following chemical-based DNA transformation method was adapted from the protocol published by Abdel-Banat et al. (Abdel-Banat, 2010 #56), to be improved for strain SD98 (U.S. patent application 62/631,541) and its derivatives, many of which are named and used in the examples described herein.

A fresh single colony of the strain to be transformed is inoculated into 5 ml TG (transformation growth medium) consisting of, per liter, 10 g yeast extract, 20 peptone, 3 g glucose, 200 mg ampicillin (sodium salt), and buffered with a final concentration of 200 mM MES (Sigma-Aldrich) adjusted to pH 6.2, with concentrated NH₄OH. This “starting culture” was grown to saturation overnight (16 to 24 hours) in a 50 mL Erlenmeyer flask at 250 rpm in a shaking incubator at 30° C. Importantly, these conditions prevent the pH of the culture from dropping below 4.5.

Strains typically grew to an OD₆₀₀ of approximately 10, during a 16 to 24 hours period. The saturated starting culture is diluted to give an OD₆₀₀ of 1.0 (typically about 1:10) into 50 ml of the same TG medium in a 500 mL Erlenmeyer flask. This culture was grown again by shaking at 250 rpm and 30° C. until it reached late logarithmic growth, an OD₆₀₀ of about 6.0 to 6.5. This typically took about 5 to 6 hours, but the time varied from strain to strain. Some of the more extensively engineered strains grow more slowly than their progenitors. A few extensively engineered strains reached saturation at an OD₆₀₀ of only 5 to 6, in which case the cells were harvested at an OD₆₀₀ of 3.0 to 3.5, aiming to harvest the cells at late-logarithmic growth.

During growth of the culture, preparations were made to ensure transformations proceeded quickly after harvest. A solution of 10 mg/mL single-stranded salmon sperm DNA (ssDNA) was prepared by heating to 100° C. in a thermocycler for 5 minutes, and then quickly chilling the tubes in an ice-water bath. 1.5 mL Eppendorf tubes were prepared for each individual transformation and chilled on ice. 10 μL of ssDNA solution were added to each of the tubes, followed by ideally 5 μL of the experimental DNA (linear or circular) destined for transformation into the strain. Sometimes, especially when multiple fragments are being introduced together, it is difficult to fit all of the DNA into 5 μL, in which case up to 10 μL of experimental DNA can be used. Ideally, the concentration of the experimental DNA should be at least 200 ng/μL, such that at least 1 μg of total transformable DNA is added per transformation tube.

Prepare a sterile Transformation Mixture (TM) to chemically prepare the cells for transformation that contains final concentrations of 40% polyethylene glycol (molecular weight about 3,000-3350, also known as PEG 3350 from Sigma-Aldrich), 0.2 M lithium acetate, 0.1 M dithiothreitol, 0.2×YPD medium (1×YPD medium is, per liter, 10 g yeast extract, 20 g peptone, and 20 g glucose). In practice, this TM is prepared by combining three stock solutions on the day of transformation. 2 M lithium acetate is made in 1×YPD medium, rather than distilled water. It is sterile filtered and stored in 1 mL aliquots at −20° C. prior to use. 1 M dithiothreitol is likewise prepared in 1×YPD medium, rather than water, and it, too, is sterile filtered and stored in 1 mL aliquots at −20° C. prior to use. Finally, a solution of 50% PEG 3350 is prepared in distilled water, sterile filtered, and stored in an air-tight container at room temperature. PEG 3350 apparently oxidizes slowly in air, during which time the pH of the solution drops. Generally, all stocks of PEG 3350 used for transformations were under 2 months old and had pH's no lower than 5.0. 10 mL preparations of “TM” were made fresh on the day of transformation by mixing together 1 mL of the sterile lithium acetate in YPD, 1 mL of the sterile dithiothreitol in YPD and 8 mL of the sterile PEG 3350 solution. The viscous TM was vortexed thoroughly to ensure proper mixing. It was then stored on ice, prior to use in the transformation.

Once the culture to be transformed reaches late logarithmic growth, cells were centrifuged at 6500 rpm for 5 minutes under conditions of gentle refrigeration (12° C.). From this point forward, the culture was handled as quickly as possible, diminishing as much as possible the time taken to get the strain to its heat shock step. The supernatant is poured away, the cell pellet is resuspended in 1 mL of TM, and centrifuged once more under the same conditions. The supernatant is removed with a micropipette. Finally, the rinsed cell pellet is resuspended in 700 μL of TM, producing a viscous cell suspension of about 900 μL to 1 mL total volume. This is suspension is sufficient for about 10 individual transformations. For all subsequent steps, the tubes are kept on ice. As quickly and carefully as possible, aliquot 85 μL portions of the cell suspension to each of the chilled transformation tubes and mix thoroughly. Together with the 15 μL of mixed DNA already present in each tube, this yields a total mixture of 100 μL per transformation. Heat shock each transformation by placing the tubes in a 42° C. water bath or heating block for 45 minutes.

After the 45 minutes heat shock, return the tubes immediately to ice, and then pellet and rinse the cells in a microfuge at full speed with 1 mL of a liquid medium version of the agar-based medium that is to be used for selection, if the selective medium is to be a drop out medium (a medium lacking a particular nutrient such as uracil. The majority of our transformations utilized URA3 or TRP5 as a selection marker, so a complete medium (with 2% Glucose), lacking (“drop out”) either uracil or tryptophan, respectively, was used to resuspend and rinse the cells. Spin down the cells of each transformation and pipette away the supernatant Resuspend each pellet in 300 μL of fresh selection medium, and spread each suspension over plates containing selective medium with 2% agar. It is normal practice to spread 1/10 of the suspension on one Petri plate, and 9/10 of the suspension on a second plate. After drying, the plates are incubated at 30° C. until colonies appear, typically in 2 to 4 days. If an antibiotic such as G418 is used for the selection, then the cells are resuspended in one ml of liquid 1×YPD medium, added to 15 ml tube containing 4 ml of liquid 1×YPD and rolled or shook at 30° C. for 3 hours. After the 3 hours, the cells are pelleted at 5,000 rpm, resuspended in 0.5 ml 1×YPD and plated on selective plates containing 1×YPD plus antibiotic. The appropriate antibiotic concentration, which is the minimum concentration necessary to eliminate any background growth of the parent strain, should be determined with a pilot experiment for each strain. Typical appropriate concentrations are 200 mg-L G418, 300 mg/L hygromycin, or 200 mg/L zeocin.

When using a linear DNA, or a mixture of linear DNA fragments to be assembled in vivo by homologous recombination of overlapping ends, when the recipient strain is Δnej1, and when the selectable gene is URA3, it is typical to obtain about 10 to 200 transformant colonies using the above method. For transformation with linear DNA that is desired to integrate into a specific chromosomal target, individual colonies are tested for the correct desired integration structure by colony PCR using the Phire Plant PCR Kit (Thermo Fisher) as instructed by the supplier. The PCR primer pairs should bracket one of the junctions between the integrated DNA and the adjacent chromosomal DNA to avoid PCR priming by sites internal to the cassette itself, which can be present as leftover from the transformation mix, or integrated at a random chromosomal location by non-homologous end joining. It is best to use two different sets of PCR primer pairs, one for each of the two junctions between the integrated cassette and the surrounding chromosomal DNA, since we have seen cases where one junction at one end of the cassette appeared to be correct, but the other end was not.

### Example 2. Construction of a Transformable Low Pyruvate D-Lactate Producing Yeast

SD1566 is an engineered derivative of a Crabtree positive strain of Kluyveromyces marxiamus that contains three integrated copies of a cassette designed to express the E. coli ldhA (EcldhA) gene. The construction and genesis of SD1566 has been described in US patent application 62-631,541, the entirety of which is hereby incorporated by reference. In SD1566, the three EcldhA cassettes are inserted at the KmPDC1, KmGPP1, and KmNDE1 loci. In all three cases, the EcldhA gene is driven by the KmPDC1 promoter, but no chromosomal sequences were deleted. SD1555, a precursor of SD1566, contained a fourth copy of EcldhA inserted at the KmPCK1 locus, but during the selection for resistance to beta-chlorolactate, which gave rise to SD1566, a spontaneous deletion of the entire KmPCK1 locus and surrounding DNA occurred, leaving SD1566 with a pck1− phenotype, which is the lack of ability to perform gluconeogenesis. Another phenotype that spontaneously arose in SD1566 was the loss of DNA transformation competence. Nonetheless, the high D-lactate productivity and low pyruvate productivity of SD1566 compelled us to discover how to further develop strains containing the desirable features of SD1566.

A precursor strain to SD1566 was SD1524, which contained the same three copies of the EcLdhA as SD1566, but SD1524 contained an intact and functional PCK1 gene, so it could grow on a minimal medium with a non-fermentable carbon source such as glycerol, D-lactate, L-lactate, or succinate as the sole carbon source. SD1524 was deleted for the KmURA3 gene, so it had an ura3− phenotype. As such, SD1566 is URA3+, pck1−, while SD1524 is ura3−, PCKJ+.

Since all of our K. marxiamus strains are assumed to contain mixtures of haploids of both mating types, we figured that SD1566 (URA3+, pck1−) could mate with SD1524 (ura3−PCK1+), and diploids could be selected by growth on uracil dropout minimal medium (Sigma Aldrich) containing 2% potassium L-lactate, pH 5.0 as the sole carbon source (CM, −ura, +L-Lac). SD1566 and SD1524 were mated by mixing the strains together in a patch on “mating medium” consisting of 2% agar and 2% Dextrose and incubating at 30° C. overnight. The mated patch was then replica plated to CM, −ura, +L-Lac plates and incubated at 37° C. After 2 days, presumably diploid strains appeared, which were streaked to produce single colonies, which in turn were patched to “sporulation medium” consisting of 2% agar, 1% potassium acetate, 0.1% yeast extract, and 0.05% dextrose. The sporulation medium plates were incubated at 30° C. for 4-7 days. Spore formation was confirmed by microscopy, and random spore analysis was performed when ˜70% or greater sporulation efficiency was seen. For the random spore analysis, a mass of cells of approximately 20 microliters was scraped with a toothpick and resuspended in 0.25 ml buffer (10 mM TrisHCl, pH 6, 1 mM Na₂EDTA), containing 200 units/ml Zymolyase (Zymo Research), with incubation at 37° C. for 45 minutes to kill vegetative cells and enrich for spores. Next, the suspension was heat treated at 57° C. for 15-25 minutes to further enrich for spores by killing vegetative cells. The spores were then serially diluted and plated on YPD agar (1% yeast extract, 2% peptone, 2% glucose, 2% agar), and resulting single colonies were checked for URA3 and PCK1 phenotypes, by plating on complete minimal medium containing uracil (Sigma-Aldrich) with 2% glucose as the carbon source, complete minimal uracil dropout medium (Sigma-Aldrich) with 2% glucose as the carbon source, or complete minimal containing uracil (Sigma-Aldrich) and 2% potassium L-lactate as the sole the carbon source.

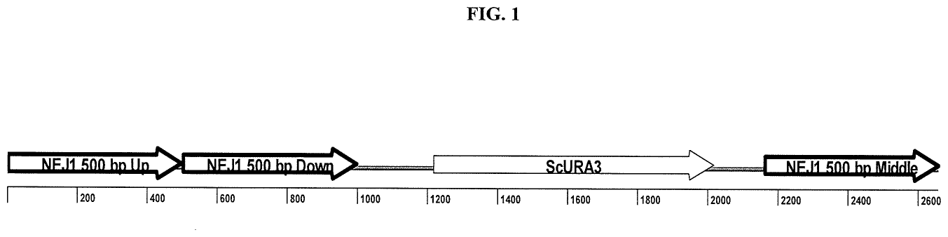

The resulting haploid-derived strains were screened for retaining the ability to produce high titers of D-lactate (more than 100 g/L), low titers of pyruvate (less than 1 g/L) as described in Example 2, ura3+, and PCK1+. One such strain, named MYR2755, was chosen for further development because it had also regained the ability to be transformed with DNA. To facilitate further engineering of MYR2755, its NEJ1 gene was deleted using the cassette shown in FIG. 1, the DNA sequence of which is given in SEQ ID No. 1. The initial transformant of MYR2755 was selected on uracil drop out medium to be URA3+, and was named MYR2785. Since MYR2785 was able to grow on a minimal medium without uracil, it was used a control parent strain in several of the examples give below. After looping out the URA3 gene by selection on 5-FOA, the Δnej1 derivative, MYR2787, which is ura3−, then became the parent for the first and subsequent installations of FFZ1 expression cassettes as described in examples given below.

### Example 3. Construction of First Generation FFZ1 Expression Cassettes in D-Lactate Producing Yeasts

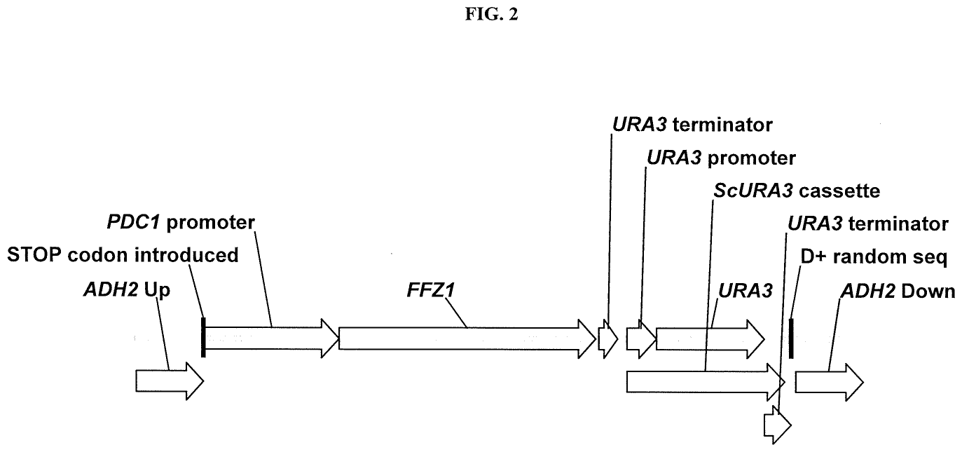

The FFZ1 gene from either Zygosaccharomyces bailii or Zygosaccharomyces rouxii was expressed by homologous integration at a genomic locus on Chromosome 4 such that the native KmADH2 gene at that locus was disrupted. The integration cassettes comprised of 1) “ADH2 up”, 501 bp DNA corresponding to the coding sequence of 1 to 501 of the native ADH2 ORF, 2) a TAA stop codon which should act as a translational stop codon to terminate translation of the partial ADH2 ORF, 3) a 1000 bp sequence containing the KmPDC1 promoter, 4) the ZbFFZ1 ORF (open reading frame) or the ZrTFZ1 ORF from Zygosaccharomyces balii or Zygosaccharomyces rouxii, respectively, 5) a ScURA3 cassette comprising of a modified Saccharomyces cerevisiae URA3 gene terminator sequence placed both upstream and downstream of a Saccharomyces cerevisiae URA3 promoter and ORF, 6) “D+”, a synthetic DNA sequence of 22 bp (AACTTAGACTAAGGAGGTTTGG), and 7) “ADH2 down”, a 500 bp sequence corresponding to coding sequence bp numbers 502 to 1001 of the native ADH2 gene. A diagram showing the structure of the ZbFFZ1 and ZrFFZ1 integration cassettes is given in FIG. 2, and the DNA sequence for the ZrFFZ1 version is given as SEQ ID No.2.

Homologous integration of the cassettes was carried out using in vivo assembly DNA fragments with overlapping homology at their ends, produced by PCR, all transformed together in roughly equimolar concentrations into the receiving strain, MYR2787, a D-lactate producing strain (See Example 2). Integrants were selected on minimal 2% glucose plates without uracil (CM without uracil, Sigma-Aldrich), substreaked on the same medium for purification, and tested by colony PCR for the desired integration event. The ScURA3 gene terminator repeats then enable a homologous “loop-out” of the ScURA3 cassette on media containing 5′-FOA (see U.S. provisional application 62/631,541) such that the FFZ1 expression cassette remains integrated and the strain becomes ura3− phenotypically, facilitating repeated use of the URA3 marker for further engineering.

Such URA3+ transformants obtained (for example SD1748 for ZbFFZ1 and SD1751 and SD1755 for ZrFFZ1 cassettes) were sequence verified and also tested in BioLector fermentations for their utilization of the sugars sucrose, glucose and fructose.

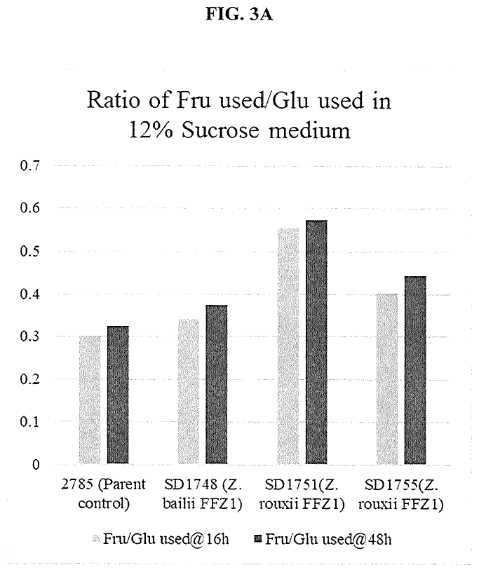

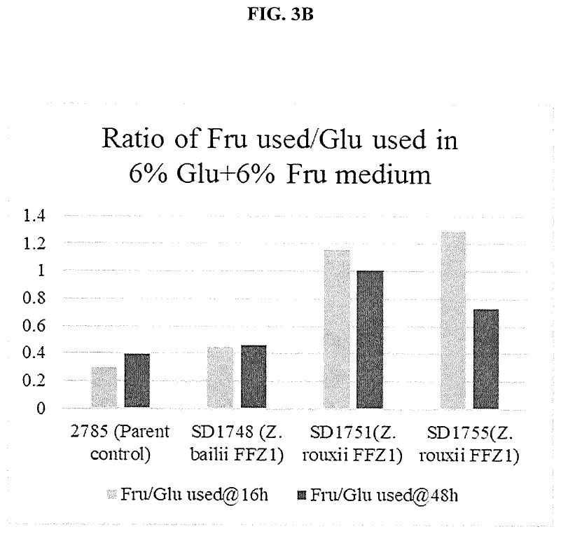

The results from such a BioLector experiment showing that the ZbFFZ1 had no significant effect and that ZrFFZ1 had a measurable effect are given in FIGS. 3A and 3B.

The BioLector experiment showed that with the ZrFFZ1 cassette installed, the utilization of fructose was improved relative to the utilization of glucose. For example, if the ratio of fructose utilized to glucose utilized is calculated at two different time points and compared with the parent strain lacking FFZ1 (MYR2785), this ratio is improved from about 0.32 to about 0.44 (12% sucrose medium, 48 hours data). This improvement is larger (from about 0.3 to about 1.3 (16 hours data) when the experiment is done using a mixture of 6% glucose and 6% fructose in the starting medium rather than using 12% sucrose in the starting medium. Taken together, these results indicate that strain SD1755 (with ZrFFZ1) was the best strain of the lot with respect to fructose utilization, so it was used for further engineering. SD1755 was compared to MYR2785 in pH-controlled 7-liter fermentors, and the fructose was again consumed earlier by SD1755 (see Example 6).

The ScURA3 gene was deleted from SD1755 by selection on 5-FOA, and the resulting ura3− derivative was named SD1774, which was capable of further engineering. It was converted from a D-lactate producer to an L-lactate producer in Example 4

### Example 4. Construction of KMS1017, an L-Lactate Producer, and KMS1019, its Ura3-Derivative Suitable for Further Engineering

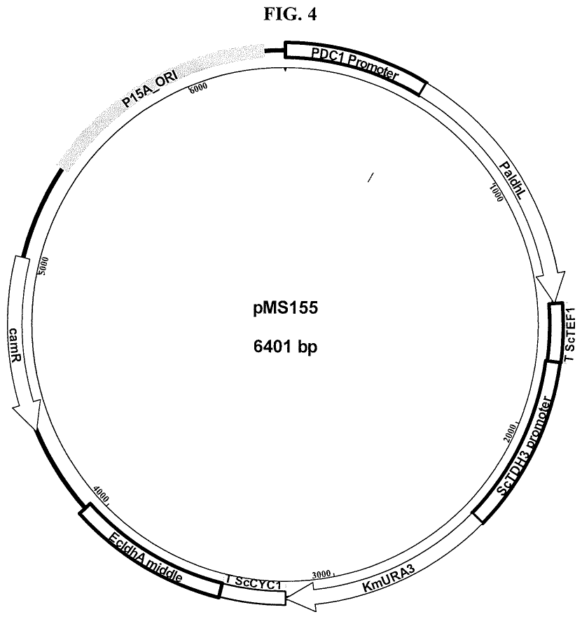

Plasmid pMS155, which was designed to contain a cassette for exchanging the PaldhL open reading frame for the EcldhA open reading frame at any of the integrated cassettes in any of the D-lactate producer strains, was constructed using the NEBuilder HiFi Assembler Cloning Kit (see FIG. 4 and SEQ ID No. 5). The PaldhL swap cassette was amplified by PCR from pMS155 and transformed into SD1774, selecting for URA3+ transformants. URA3+ colonies were then tested by colony PCR to determine which of the three copies of the EcldhA gene had been replaced. Strain KMS977 was shown to have the copy at the GPP1 locus replaced. The URA3 gene in KMS977 was then deleted by homologous recombination and selection on medium containing 5′-FOA, producing KMS984. KMS984 was subsequently transformed with the same swap cassette, this time replacing EcldhA at the NDE1 locus to give KMS1001. The URA3 gene in KMS1001 was then deleted by selection on medium containing 5′-FOA, producing strain KMS1004. KMS1004 was transformed with the swap cassette, exchanging the last the remaining EcldhA gene at the PDC1 locus to produce KMS1017, which produces only L-lactate. The URA3 cassette was deleted from KMS1017 as described above yielding KMS1019. KMS1017 contains only PaldhL cassettes, so it produces only L-lactate. KMS1019 is ura3−, so it is set up for further genetic engineering, as in Example 5.

### Example 5. Redesigned Integration and Improved Function of the Zygosaccharomyces Rouxii FFZ1 Gene

Given the modest, but measurable, increase in fructose utilization relative to glucose utilization resulting from inserting one copy of the ZrFFZ1 expression cassette gene into the middle of KmADH2 on chromosome 4, it was desirable to further improved fructose utilization by adding an additional copy of a ZrFFZ1 expression cassette to our existing D-Lactate production strains SD1755 and MYR2785. The possibility existed that ZrFFZ1 was simply unable to replicate its normally powerful function of fructose permeability when expressed in a heterologous host. To test that theory, the ZrFFZ1 expression cassette was redesigned in two new ways, in an attempt to find a method of integration that would increase its effectiveness in K. marxianus.

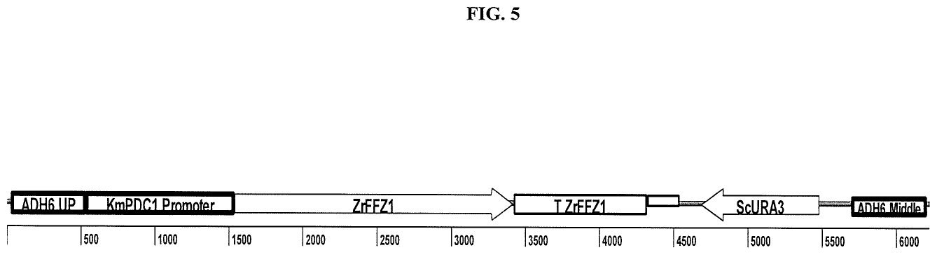

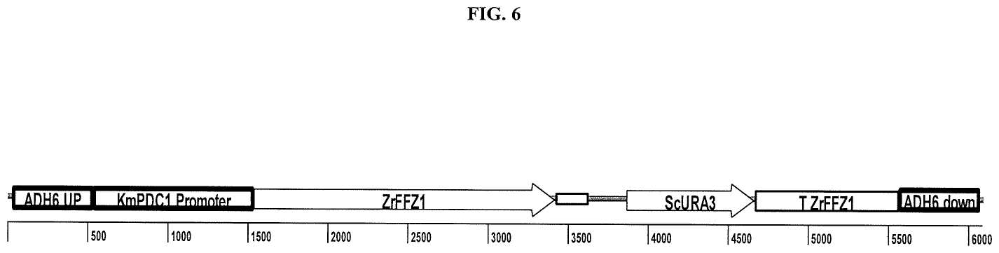

Two new integration cassettes were designed to express ZrFFZ1 in K. marxianus. One, named JSS89, was designed delete and replace the entire ORF of KmADH6. The other, named JSS90, was designed to insert into the middle of the KmADH6 ORF. The structures of the two cassettes, JSS89 and JSS90, are shown in FIGS. 5 and 6, respectively. The DNA sequences of these two cassettes are given in SEQ ID No. 3 and SEQ ID No. 4 respectively. The same 1,000 base pair KmPDC1 promoter (P_{PDC1 1,000 bp}), preceded by a TAA stop codon, that had been used in the cassette design of Example 2, was again used to drive ZrFFZ1 expression from both of these new cassettes. Both new cassettes differ from the earlier design used to create SD1755 in Example 2, however, in that both JSS89 and JSS90 utilize the ZrFFZ1 gene's native terminator sequence from Zygosaccharomyces rouxii Saccharomyces cerevisiae URA3 was used as the selection marker in both cases, but it was installed differently in the two cassettes to facilitate integration into our lactate production strains. The pJSS89 cassette expressed ScURA3 in the opposite direction of the FFZ1 gene, such that the cassette would be unable to integrate within the DNA “scars” left behind at earlier integration events in our strain lineages. The pJSS90 cassette avoided the same problem by expressing a slightly shortened ScURA3 gene that lacked its native terminator, directly in line after a copy of the ZrFFZ1 ORF and 207 base pairs of the native ZrFFZ1 terminator. The ScURA3 promoter and ORF was followed by the entire 900 bp Zr FZ1 terminator, used here to terminate the ScURA3 transcript, as well provide a direct repeat sequence of 207 base pairs that would permit the removal of the marker gene by 5-FOA selection.

In cassette JSS89, the entire ZrFFZ1-900 bp terminator was followed by a 220 bp sequence corresponding to the native terminator for KmADH6. This was followed by the complete, inverted ScURA3 marker, and then by a 500 bp fragment of DNA corresponding to the center of the KmADH6 sequence. This fragment, appearing natively 270 bp downstream from the ATG of KmADH6, acts as a “downstream” integration flank for the cassette. Preceding the TAA-P_{PDC1-1,000bp} Promoter construct at the front of this cassette, was a 500 bp fragment of DNA corresponding to the upstream regulatory region of KmADH6 (−510 bp to −10 bp 5′ to the ATG). This acted as an “upstream” integration flank for the entire cassette.

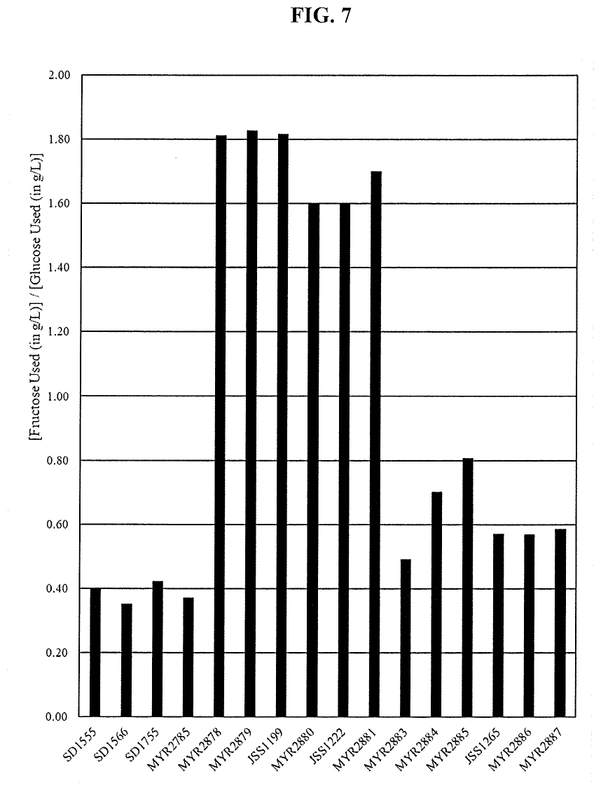

The DNA fragments (with 60 bp regions of overlapping DNA homology with each adjacent fragment) needed to build the JSS89 and JDSS90 integration cassettes were produced by PCR, and mixed in equimolar amounts. This mixture was then transformed by the method described above into Δura3 derivatives of SD1755 (called SD 1774) and MYR 2785 (called MYR 2787), and the overlapping fragments joined together and integrated by homologous recombination. URA3⁺ transformant clones were screened by colony PCR to identify correct integrations of the cassettes from FIGS. 5 and 6 into each strain. Fermentations of each clone, in multiple isolates, were conducted using SDM2 minimal medium, containing 12% Sucrose and 150 mM Ammonium Phosphate Buffer, pH 6.3, in a BioLector Basic (sold by m2p Labs). Strains were cultured until growth ceased due to low pH, 75 hours. Samples were analyzed by HPLC for D-lactate (FIG. 7).

As expected from the earlier results, adding an additional copy of ZrFFZ1 further increased the rate of fructose utilization in all strains. The pJSS90 cassette behaved in a roughly equivalent fashion to the previous insertion cassette, which had been integrated at KmADH2 in SD1755. Each increased the ratio of fructose used to glucose used by approximately 0.1 to 0.2, compared to the parent strains (FIG. 7). The contribution from the two integrated cassettes was approximately additive, as the integrants of the pJSS90 cassette into SD1774 were commensurately higher, with respect to the ratio of fructose used to glucose used, than similar integrants made into MYR2787.

Unexpectedly, however, there was a dramatic difference in performance between the two cassettes designed to install the ZrFFZ1 gene into ADH6. Whereas integrating JSS90 into MYR2787 increased the ratio of fructose used to glucose Used from 0.35 to 0.55, adding one copy of the JSS89 cassette increased it all the way to 1.65, a roughly 6-fold increase in effectiveness compared to either of the previous insertion cassettes (FIG. 7). This single, dramatic increase was more than sufficient on its own to completely change the metabolism of the strain, from glucophilic to fructophilic. Interestingly, this effect was also additive to that of the initial ZrFFZ1 cassette at ADH2, though the effect of the initial cassette seemed to remain much more minor, as having both cassettes present increased [Fructose Used][Glucose Used] to only 1.85 from 1.65.

In each case, adding further copies of ZrFFZ1 to these engineered strains of Kluyveromyces marxianus enhanced the originally glucophilic strain s ability to absorb and metabolize fructose during growth. The effect is pronounced, however, not just according to this specific heterologous allele in Km, but also due to the circumstances of its expression. Integrating ZrFFZ1 in place of KmADH6, while using an extended terminator sequence of native Zr DNA following the gene (900 bp), enabled a large and unanticipated boost in fructose utilization. This boost was consistently seen in fermentations of different sizes and lengths, as new Km D-Lactate producing strains (listed in Table 2.) retained their fructophilic character even in base-controlled 7 L Fermentations that ran to completion (see Example 6).

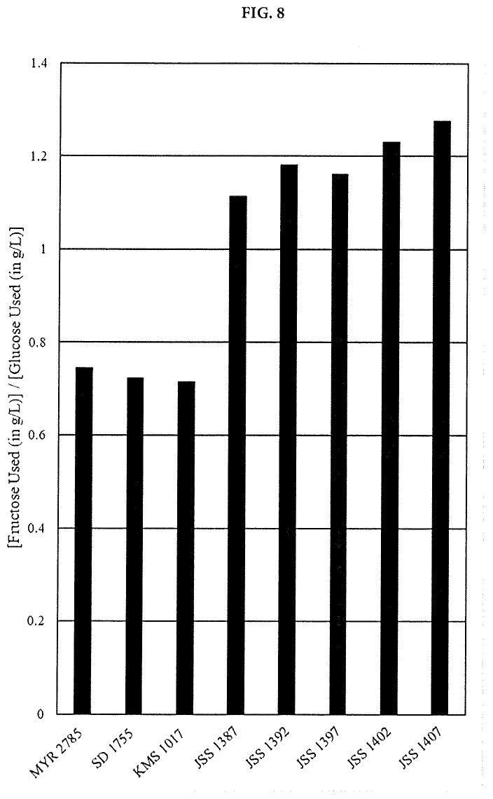

The character-changing ability of the JSS89-based “replacement” cassette for ZrFFZ1 expression functioned similarly when it was installed in an L-lactate producing strain KMS1017 derivative KMS1019 (see Example 4). When added to KMS1019, the Δura3 derivative of KMS1017, a similar improvement in fructose utilization was obtained. A summary of five isolates obtained by transforming the JSS89 cassette into KMS1019 is given in Table 3. All of the new strains showed an increased fructophilic character in shake flask fermentations that contained calcium carbonate to hold the pH steady near 6 for the early part of the fermentation (FIG. 8). Note that the observed magnitude of the improved fructose utilization is lower in shake flasks than in the BioLector fermentations described above. The best derivatives of KMS1019 that contained the JSS89 cassette were evaluated in 7-liter fermentations in which the pH was controlled by feeding calcium hydroxide. In the 7-liter fermentations, the best performing isolate was JSS1397 (see Example 6).

### Example 6. Production of L-Lactate or D-Lactate by Strains Containing a ZrFFZ1 Cassette in pH-Controlled 7-Liter Fermentors

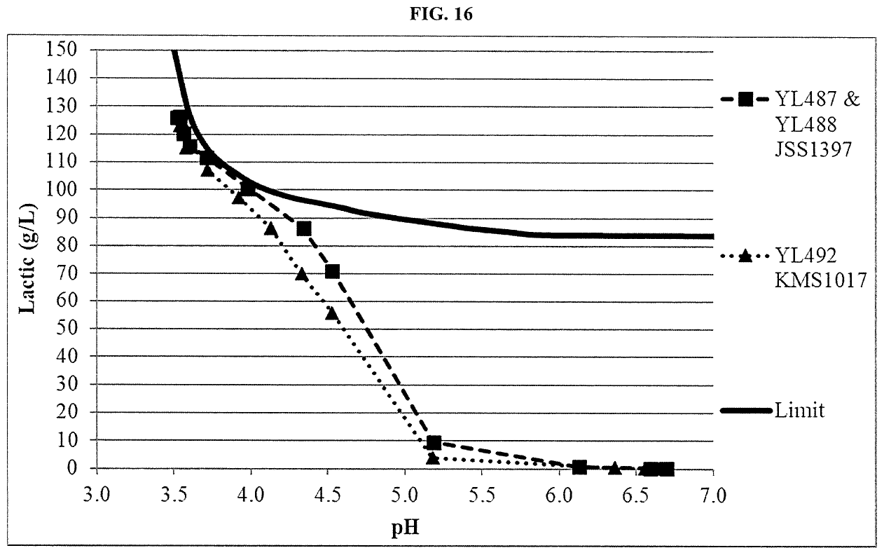

Inocula of yeast strain JSS1397 were grown at 37° C. in 150 ml of YPS-MES medium (see Table 4) in 500 ml baffled shake flasks to an OD 600 nm of 3.0 to 4.0. 150 ml was inoculated into 4 liters of AM1S medium (see Table 4). Impeller speed was 750 rpm and aeration was 300 ml/min, equal to 0.075 vvm of the starting volume. The starting pH was about 6.8. pH was controlled by automatically controlled peristaltic pumping of a slurry of 3 molar calcium hydroxide, which was kept suspended in a stirred reservoir. The pH set point was automatically ramped down (i.e., decreased) to pH 4.25 in a linear fashion from time zero (inoculation time) to 25 hours. At 25 hours, the set point was changed to pH 3.5. This allowed the pH to fall naturally as more L-lactic acid was produced. At the end of the 45 hour fermentations, the final pH was 3.5. The pH ramp prevented the precipitation of calcium lactate. In FIG. 16 the solubility of calcium lactate is shown as a function of pH.

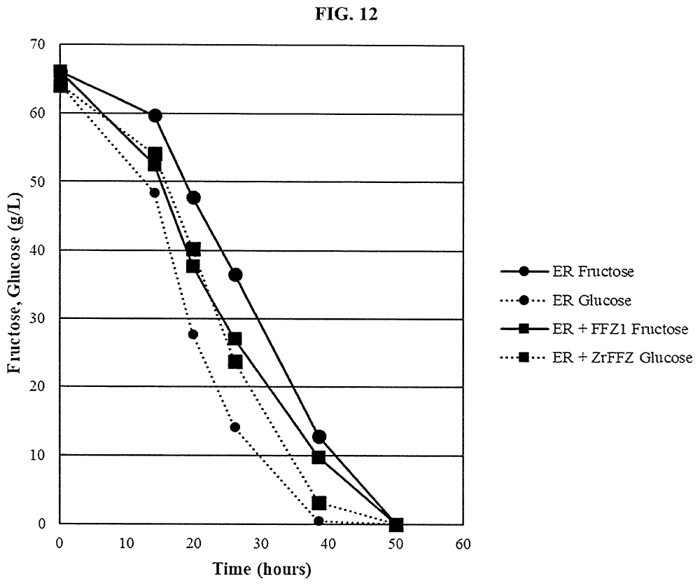

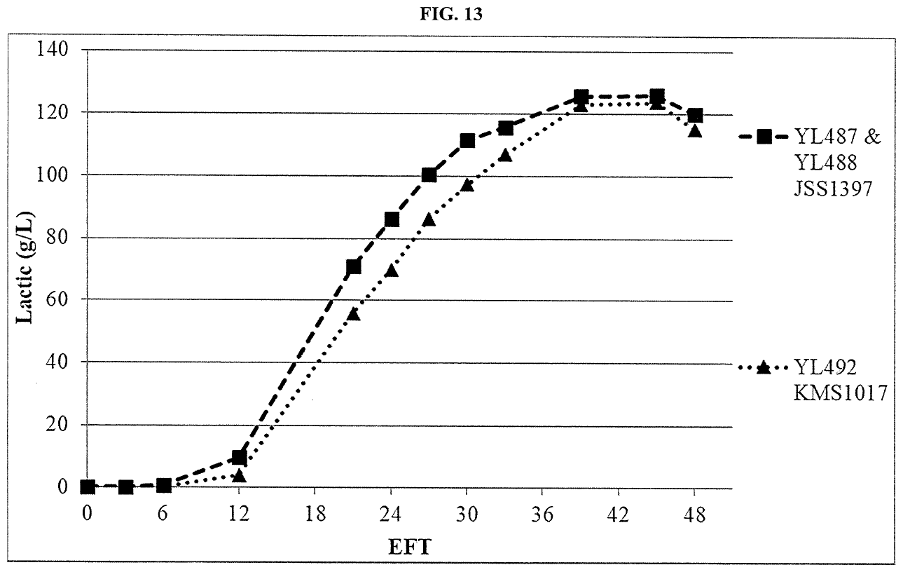

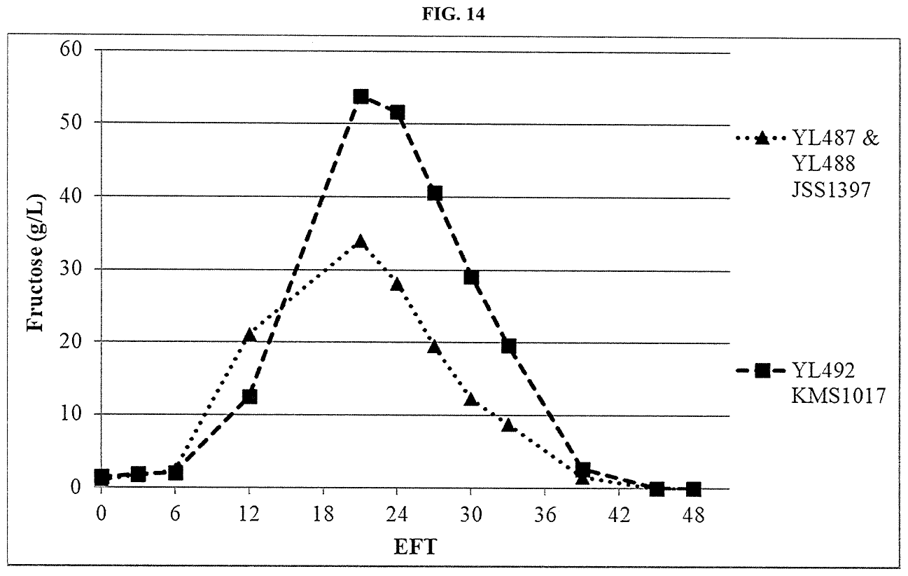

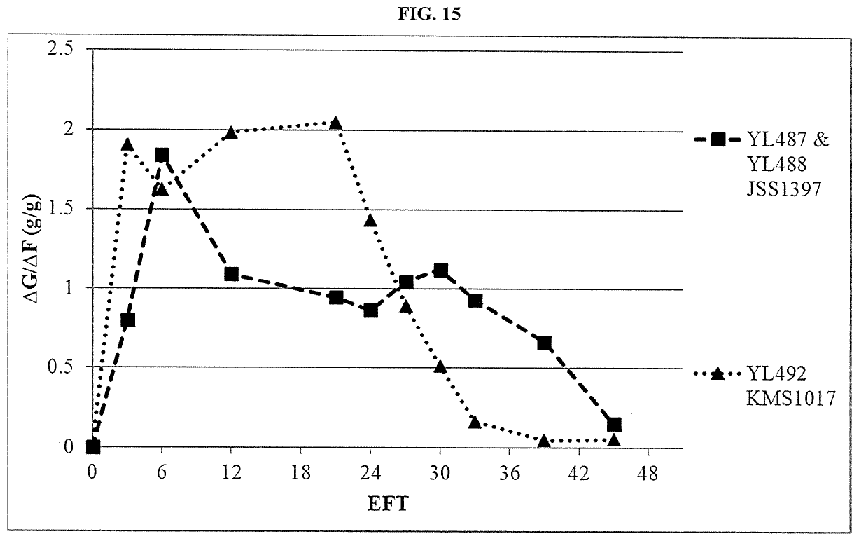

At 45 hours the L-LAC titer 126 g/L and the calculated yield was 0.85 μg sucrose (averages of duplicate fermentations, named YL487 and YL488 in the FIGS. 12-16). The maximum specific productivity for L-LAC was 3.75 g/L-hr and the cumulative productivity was 3.4 g/L-hr. Although the final L-lactate titer of JSS1397 was similar to that of the control strain KMS1017 (named fermentation YL492 in FIGS. 13-16), the rate of production by JSS1397 was much faster, (FIG. 13). The increased rate of fructose use can be seen by measuring the free fructose after sucrose hydrolysis (FIG. 14). Even more dramatic is the ratio of glucose to fructose use shown in FIG. 15. The ratio of the rates of glucose to fructose utilization for JSS1397 was approximately the same for much of the fermentation whereas the control strain KMS1017 used glucose at twice the rate of fructose early in the fermentation, and only used glucose and fructose at the same rate briefly at 27 hours EFT (elapsed fermentation time) when the ratio of fructose to glucose remaining was 4:1.

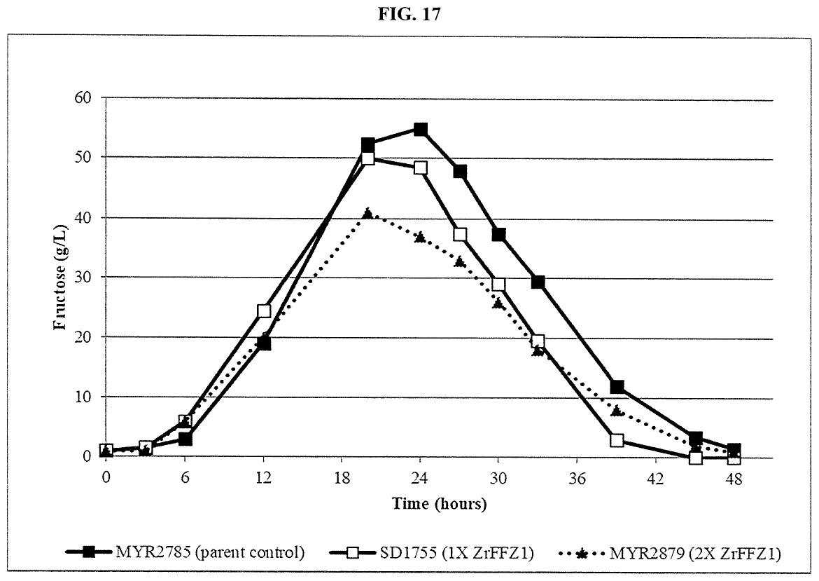

In similar 7-liter fermentations, D-lactate producing strains SD1755 (contains one cassette of ZrFFZ1), MYR2879 (contains two cassettes of ZrFFZ1) or MYR2785 (without a ZrFFZ1 cassette) were compared. In this case, the starting sucrose concentration was slightly lower at 180 g/L. As shown in FIG. 17, the rate of fructose consumption was increased by both MYR2879 and SD1755 than by MYR2785. Moreover, the MYR2879 showed the earlier fructose consumption than that of SD1755. The final fermentation parameters obtained for this comparison is given in Table 5.

### Example 7. Solving Fructose Problem in the Canejuice Fermentation for L-Lactate

In many regions where the climate is tropical or warm, sugar cane is a preferred source of fermentable sugar. After harvesting, the cane is mechanically breaking and milled to extract the juice, and then the canejuice is purified in several steps to make sugars. The predominant sugar in canejuice is sucrose. In commercial production, canejuice is preferred to use as the low cost feedstock to produce several bio-based chemical. K. marxianus secretes an enzyme that hydrolyzes sucrose into glucose and fructose outside of the cell membrane, and then the glucose and fructose are imported into the cell where they enter the glycolytic pathway. When the yeast K. marxianus is grown in a canejuice that contains high concentrations of sucrose, the production of extracellular glucose and fructose by invertase exceeds the cells' capacity to import the glucose and fructose, so glucose and fructose accumulate outside the cells. As the fermentation proceeds, the glucose is used more rapidly than the fructose, so the fermentation time must be extended in order for all of the fructose to be consumed. This phenomenon is called the “fructose problem” which is clearly illustrated in the case of L-lactate production as following experiment.

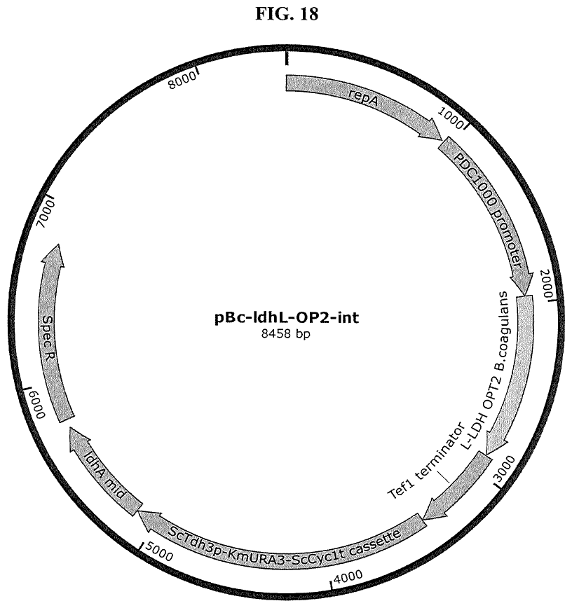

To illucidate the phenomenon, the new L-lactate producing strain without ZrFFZ1 cassette was constructed. The lactate dehydrogenase gene from Bacillus coagulans BC060 was selected to express in K. marxianus yeast. Plasmid pBc-ldhL-OP2-int, which was designed to contain a cassette for exchanging the BcldhL open reading frame for the EcldhA open reading frame at any of the integrated cassettes in any of the D-lactate producer strains, was constructed using the NEBuilder HiFi Assembler Cloning Kit (see FIG. 18 and SEQ ID No. 7). The BcldhL swap cassette was amplified by PCR from pL-BCldh and transformed into MYR2787, selecting for URA3+ transformants. URA3+ colonies were then tested by colony PCR to determine which of the three copies of the EcldhA gene had been replaced. Strain MYR2891 was shown to have the copy at the GPP1 locus replaced. The URA3 gene in MYR2891 was then deleted by homologous recombination and selection on medium containing 5′-FOA, producing MYR2891-ura. MYR2891-ura was subsequently transformed with the same swap cassette, this time replacing EcldhA at the NDE1 locus to give MYR2892. The URA3 gene in MYR2892 was then deleted by selection on medium containing 5′-FOA, producing strain MYR2892-ura. MYR2892-ura was transformed with the swap cassette, exchanging the last the remaining EcldhA gene at the PDC1 locus to produce MYR2893, which produces only L-lactate. MYR2893 contains only BcldhL cassettes, so it produces only L-lactate without the cassette of ZrFFZ1.

L-lactate fermentation of JSS1397 (with ZrFFZ1 cassette) and MYR2893 (without ZrFFZ1) were conducted in pH controlled 5-liter fermentors. Inocula of yeast strain JSS1397 and MYR2893 were grown at 37° C. in 150 ml of YPS-MES medium (see Table 4) in 500 ml baffled shake flasks to an OD 600 nm of 3.0 to 4.0. 150 ml was inoculated into 3 liters of AM1CJ medium (see Table 4). Impeller speed was 750 rpm and aeration were 195 ml-min, equal to 0.065 vvm of the starting volume. The starting pH was about 6.8. pH was controlled by automatically controlled peristaltic pumping of a slurry of 3 molar calcium hydroxide, which was kept suspended in a stirred reservoir. The pH set point was automatically ramped down (i.e., decreased) to pH 4.1 in a linear fashion from time zero (inoculation time) to 25 hours. At 25 hours, the set point was changed to pH 3.5. This allowed the pH to fall naturally as more L-lactic acid was produced. The fermentation experiments of each strain were duplicate fermentations and the average results were summarized in Table 6.

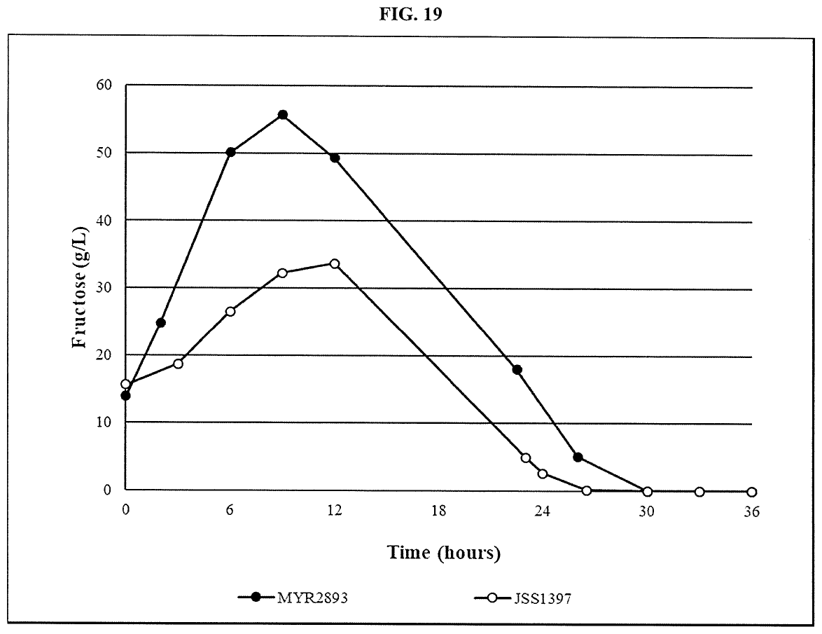

As shown in FIG. 19, the free fructose in the JSS1397 culture during the fermentation was dramatically lower than that of MYR2893. Moreover, the fructose in JSS1397 culture was completely consumed within 27 hours with fructose consumption rate of 3.66 g L¹ hr¹. This was earlier than that of MYR2893 in which the fructose was completely consumed at 30 hours with fructose consumption rate of 3.28 g L¹ hr¹. This experiment clearly demonstrated that the cell with ZrFFZ1 cassette can solve the “fructose problem” in canejuice fermentation for L-lactate production.

### Example 8. Solving the Fructose Problem in Saccharomyces cerevisiae Ethanol Fermentations

Fermentation of sugar derived from cane juice by Saccharomyces cerevisiae yeast strains into ethanol for fuel and beverage use is practiced on a large scale. Like K. marxianus, S. cerevisiae secretes an enzyme that hydrolyzes sucrose into glucose and fructose outside of the cell membrane, and then the glucose and fructose are imported into the cell where they enter the glycolytic pathway. In the case of S. cerevisiae, the secreted enzyme is named invertase or sucrase or sucrose hydrolase, among other names. When S. cerevisiae is grown in a medium that contains high concentrations of sucrose, the production of extracellular glucose and fructose by invertase exceeds the cells' capacity to import the glucose and fructose, so glucose and fructose accumulate outside the cells. As the fermentation proceeds, the glucose is used more rapidly than the fructose, so the fermentation time must be extended in order for all of the fructose to be consumed. This phenomenon is illustrated in the following experiment.

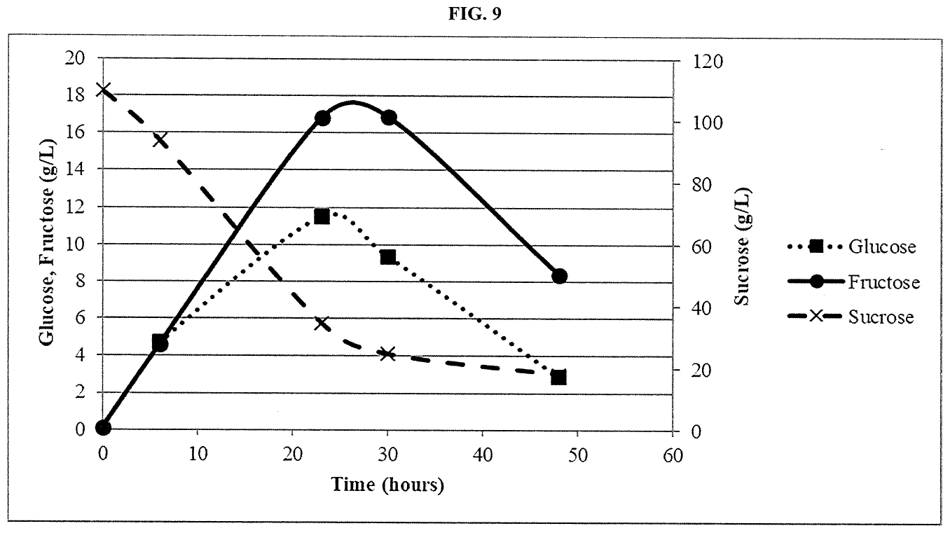

The commercially available distillery strain Ethanol Red (LaSaffre Advanced Fermentations) was grown in microaerobic shake flasks (100 ml in a 250 ml Erlenmeyer, 80 rpm, no sloshing) at 34° C. in a medium comprised of 2×Yeast Nitrogen Base (Sigma-Aldrich) and 12% w/v sucrose. Sucrose, glucose, and fructose concentrations were measured by HPLC as a function of time (FIG. 9). At the end of the fermentation, there was substantially more fructose than glucose remaining, so it was clear that Ethanol Red has the “fructose problem”.

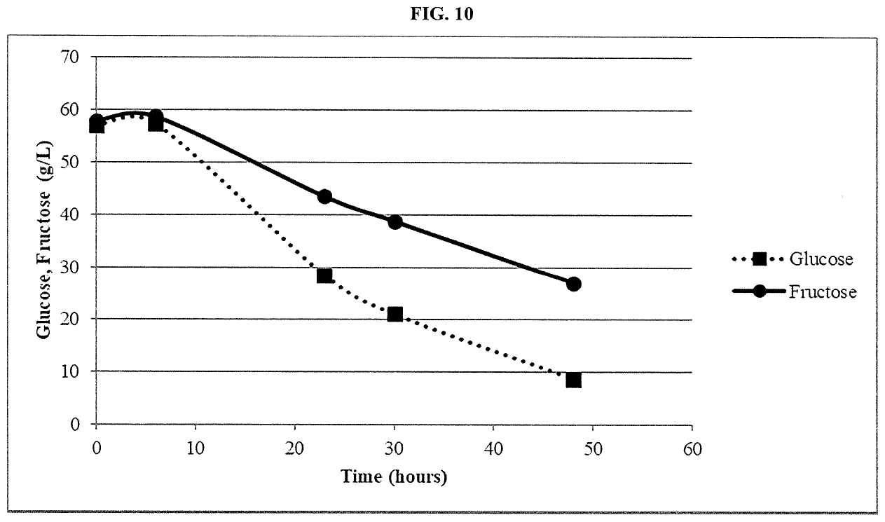

Often the sucrose in cane juice becomes hydrolyzed during treatment and/or storage. For example, in the production of high test molasses, cane juice is heated and water is evaporated to form a concentrated mix. As a result of exposure to the heat, the slightly acidic pH, and possibly due to enzymes, a large portion of the sucrose becomes hydrolyzed to glucose plus fructose. To simulate a medium made from such a mix, the above experiment was repeated, except that the medium was 2×Yeast Nitrogen Base plus 6% w/v glucose and 6% w/v fructose. The results are shown in FIG. 10. Again, at the end of the fermentation, there remains more fructose than glucose, so the “fructose problem” exists not only when the stating medium contains sucrose, but also when the starting medium contains mixtures of glucose and fructose.

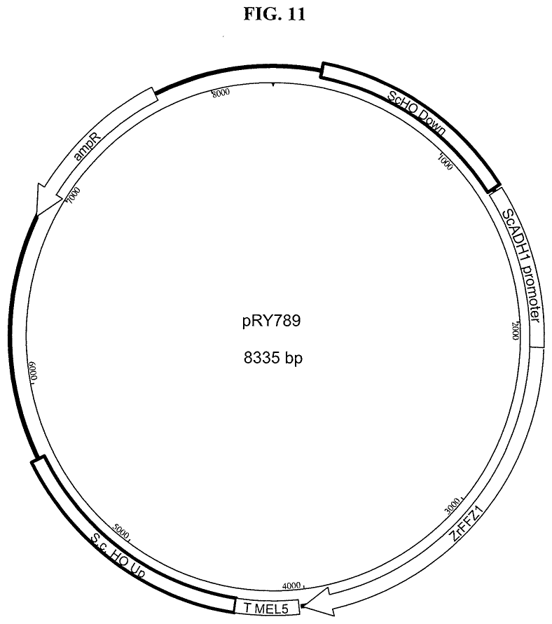

It was next shown that the fructose problem in S. cerevisiae could be solved by installing the ZrFFZ1 gene in an expression cassette. For expression of ZrFFZ1 in S. cerevisiae, a cassette was constructed that expresses the ZrFFZ1 open reading frame from the strong constitutive ScADH1 promoter, with a transcription terminator from the S. cerevisiae MEL5 gene. The cassette was designed to integrate at the HO locus by installing flanking DNA sequences of about 1 kb that are homologous to the HO locus. All of the component DNA sequences described above were generated by PCR using Phusion High Fidelity PCR Master Mix (New England Biolabs according to the manufacturers protocol. The component DNA sequences were assembled into a plasmid that replicates in E. coli named pRY789, using the NEBuilder HiFi DNA Assembly Cloning Kit (New England Biolabs) according to the manufacturers protocol. The structure of pRY789 is shown in FIG. 11, and the DNA sequence of the entire pRY789 is given as SEQ ID No. 6. The expression cassette was integrated at the HO locus of Ethanol Red by methods well known in the art for S. cerevisiae, a combination of making transformation competent cells and co-transforming the linear cassette DNA with a replicating plasmid providing antibiotic G418 resistance (Gietz, 2014 #63); Rudolph, 1985 #80; U.S. Pat. No. 6,214,577). A correct integrant was identified by diagnostic PCR on individual colonies using Phire Plant PCR Master Mix (ThermoScientific) according to the supplier's protocol.

The resulting strain, named ER+ZrFFZ1, was compared to the parent strain Ethanol Red (ER) in microaerobic shake flasks as described above, with a medium comprised of 2× Yeast Nitrogen Base with 6% glucose and 6% fructose, except in this experiment, the temperature was lowered to 26° C. in order to be able to conveniently collect more data at intermediate times before sugars were completely consumed. As shown in FIG. 12, the engineered strain ER+ZrFFZ1 used fructose more quickly, and glucose more slowly, than the parent strain. The improvement in fructose utilization by ER ZrFFZ1 was modest but significant. The data shown is the average of duplicate flasks for Ethanol Red and the average of triplicate flasks for ER+ZrFZ1. The net result is that fructose and glucose were used at similar rates by the engineered strain, such that both sugars were completely consumed at about the same time. The pattern seen here is similar to that of our K. marxiamus strains engineered for D-lactate or L-lactate production described above.

### Example 9. Increasing the Rate of Phosphorylation of Fructose

Having addressed the bottleneck of importing fructose into the cell, the next potential rate limiting step is the phosphorylation of fructose to produce fructose-6-phosphate by an enzyme such as fructokinase (EC 2.7.1.4) or hexokinase (EC 2.7.1.1). K. marxianus has two native hexokinase genes, GLK1 and RAGS. The encoded enzymes phosphorylate both glucose and fructose. Plants such as Arabidopsis thaliana (thale cress) and Solanum lycopersicum (tomato) have well characterized genes, for example AtFRK1-7 and SlFRK1-4, that encode dedicated fructokinases (Stein, 2018 #88). Intron-free open reading frames from any of these genes can be obtained by PCR (from the K. marxianus genome for KmGLK1 and KrRAG5, or from a cDNA clone for the plant genes), or the open reading frames (minus any organelle targeting sequences) can be synthesized from gBlocks (Integrated DNA Technologies) and expressed from a strong constitutive promoter in yeasts, such as the KmPDC1 promoter in K. marxianus in order to increase the flux of fructose into the glycolytic pathway. The expression cassettes were integrated into L-lactate producing yeasts in which the non-essential open reading frame of the gene at the targeted chromosomal integration site were precisely deleted. The L-Lactate producing yeasts, KMS1019 (KMS1017 ura), JSS1398 (JSS1397 ura), and JSS1408 (JSS1407 ura), were used as parental strains. As results, the 35 recombinant yeasts were constructed as listed in Table 7. Each recombinant was determined the performance of L-LAC fermentations as well as the sugar consumptions in pH controlled 5-liter fermentors as the same experimental methods described above in Example 7 with two modifications. First the AMIS medium with 150 g/L sucrose was used in this experiment. Second, the pH was set up to 3.5 at the beginning of fermentation. This allowed the pH to fall naturally as more L-lactic acid was produced.

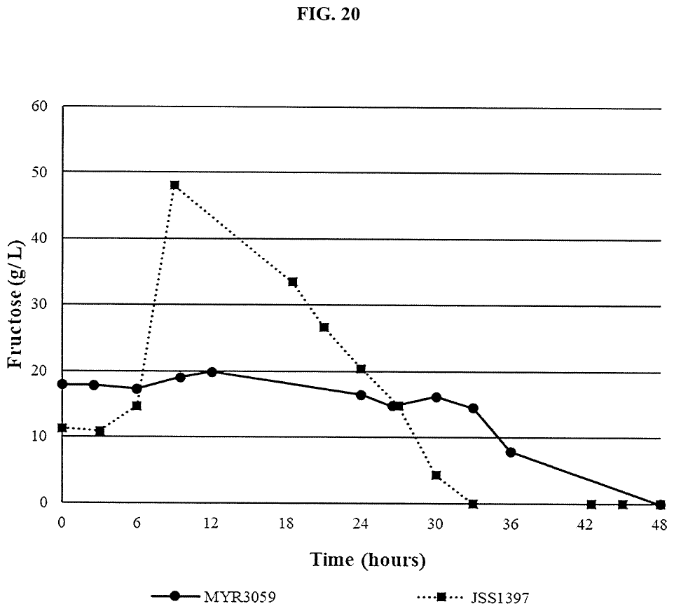

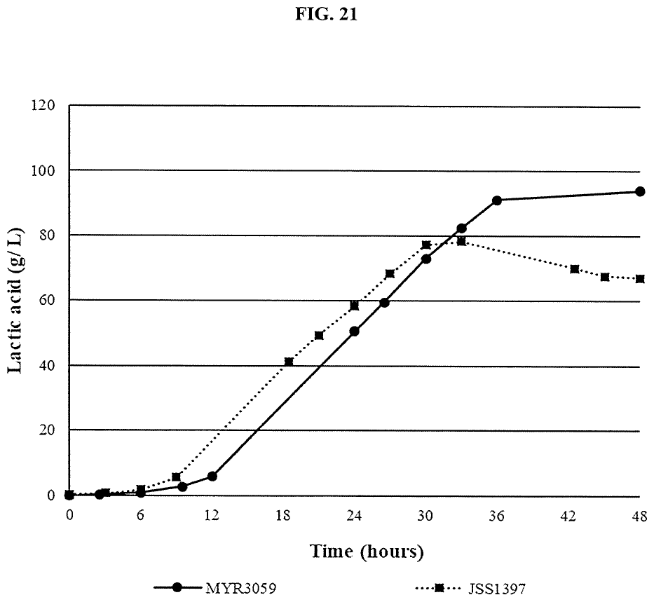

During the experiment, the inventors found the unexpected behaviors of the cells containing KmRAG5 derived from JSS1408, so called MYR3058 and MYR3059. The free fructose in the culture of both recombinant yeasts dramatically reduced comparing to that of parental strain. To confirm the behavior, the MYR 3059 was selected to determine the fermentation performance comparing to the JSS1397 which have similar genetic background to JSS1407 (JSS1408 URA⁺) and was used to demonstrate the performance along this invention. The fermentation performances were conducted in pH controlled 5-liter fermentors as the same experimental methods described above in this Example. The results illustrate that the yeast cells having fructokinase gene (RAGS had an improvement of fructose consumption as shown in FIG. 20 and the performance of L-LAC production of these cell also improved as shown in FIG. 21.

### Example 10. Increasing the Rate of Phosphorylation of fructose-6-phosphate to Produce fructose-1, 6-bisphosphate

The next step after fructokinase in the metabolism of fructose is a second phosphorylation to give fructose-1, 6-bisphosphate, by the enzyme phosphofructokinase 1 (EC 2.7.1.11). In K. marxianus, the wild type phosphofructokinase 1 is octameric and comprised of four copies each of two non-identical subunits encoded by the KmPFK1 and KmPFK2 genes. The enzyme is allosterically inhibited by ATP. It is known in S. cerevisiae, which has a similar enzyme, how to create a mutant version that is hyperactive and resistant to inhibition by ATP (Lobo, 1982 #98; (odicio, 2000 #113). This mutant version can be transplanted by straightforward genetic engineering into a K. marxianus strain to increase flux through glycolysis for the purpose of increasing production of desired chemicals such as L-lactate or D-lactate. In addition, similar mutations can be installed in the K. marxianus PFK genes or homologs thereof. Such mutations, especially in strains with increased capacity for fructose import and/or fructokinase activity, are useful for increasing flux to desired products that are derived from the glycolytic pathway, which includes products from the tricarboxylic acid cycle.

From the results of all Examples described above reflect that the genetically engineered yeast cell with comprises at least one heterologous DNA cassette that function as a fructose importer which was discovered in this invention having improvement of fructose utilization as mentioned in the summary of the invention.

TABLE 1
Gene names and descriptions

| Gene | | | Other names |
|---|---|---|---|
| name | | | encountered in |
| used | Protein or function | | literature (not |
| herein | encoded | Source organism | comprehensive) |
| EcldhA | D-lactate | E. coli | ldhD, D-ldh |
| | dehydrogenase | | |
| PaldhL | L-lactate | Pediococcus | L-ldh |
| | dehydrogenase | acidilacti | |
| BcldhL | L-lactate | Bacillus coagulans | L-ldh |
| | dehydrogenase | | |
| URA3, | Orotidine-5′- | K. marxianus, | |
| ScURA3, | phosphate | S. cerevisiae | |
| KmURA3 | decarboxylase | | |
| PDC1, | Pyruvate | K. marxianus | PDC, pdc |
| KmPDC1 | decarboxylase | | |
| GPP1, | Glycerol-3-phosphate | K. marxianus | |
| KmGPP1 | phosphatase | | |
| PCK1, | Phosphoenolpyruvate | K. marxianus | PCK, pck |
| KmPCK1 | carboxykinase | | |
| NDE1, | NADH | K. marxianus | |
| KmPCK1 | dehydrogenase-1 | | |
| IEF1, | Translation | Ashbya gossypii, | eft, eftu, eftU |
| AgTEF1, | elongation factor 1 | K marxianus, | |
| KmTEF1, | | S. cerevisiae | |
| ScTEF1 | | | |
| CYC1, | Cytochrome C | S. cerevisiae, | |
| ScCYC1, | | K. marxianus | |
| KmCYC1 | | | |
| ZbFFZ1 | Fructose facilitator | Zygosaccharomyces | FFZ1 |
| | | bailii | |
| ZrFFZ1 | Fructose facilitator | Zygosaccharomyces | ffzi1 |
| | | rouxii | |
| GLK1, | Fructokinase | K. marxianus | |
| RAG5 | | | |
| FRK1, | Fructokinase | Tomato | |
| FRK2 | | | |
| FRK1, | Fructokinase | Thale cress | |
| FRK2 | | | |

TABLE 2
Summary of D-lactate strains containing
integrated ZrFFZ1 cassettes

| Strain | ZrFFZ1 cassette | Integration | Parent |
|---|---|---|---|
| Name | integrated | type | Strain |
| MYR 2878 | JSS89 | ADH6 deletion | SD 1774 |
| MYR 2879 | JSS89 | ADH6 deletion | SD 1774 |
| JSS 1199 | JSS89 | ADH6 deletion | SD 1774 |
| MYR 2880 | JSS89 | ADH6 deletion | MYR 2787 |
| JSS 1222 | JSS89 | ADH6 deletion | MYR 2787 |
| MYR 2881 | JSS89 | ADH6 deletion | MYR 2787 |
| MYR 2883 | JSS90 | ADH6 insertion | SD 1774 |
| MYR 2884 | JSS90 | ADH6 insertion | SD 1774 |
| MYR 2885 | JSS90 | ADH6 insertion | SD 1774 |
| JSS 1265 | JSS90 | ADH6 insertion | MYR 2787 |
| MYR 2886 | JSS90 | ADH6 insertion | MYR 2787 |
| MYR 2887 | JSS90 | ADH6 insertion | MYR 2787 |

TABLE 3
Summary of L-lactate strains containing
integrated ZrFFZ1 cassettes

| Strain | Parent | Cassette | Cassette |
|---|---|---|---|
| Name | Strain | Integrated | Type |
| JSS 1387 | KMS 1019 | JSS 89 | ADH6 deletion |
| JSS 1392 | KMS 1019 | JSS 89 | ADH6 deletion |
| JSS 1397 | KMS 1019 | JSS 89 | ADH6 deletion |
| JSS 1402 | KMS 1019 | JSS 89 | ADH6 deletion |
| JSS 1407 | KMS 1019 | JSS 89 | ADH6 deletion |

TABLE 4
Composition of growth media. All amounts listed are
per liter. For petri plates, 20 g/L agar was added

| | CM minus | | | | | | | | | YPS- | |
|---|---|---|---|---|---|---|---|---|---|---|---|
| Ingredient | uracil | SDM2 | | AM1S | | AM1CJ | | YPD | | MES | |
| Glucose | | variable | | | | | | 20 g or | | | |
| | | (20-200 g) | | | | | | variable | | | |
| Sucrose | | variable | | 185 g or | | | | | | 20 | g |
| | | (20-200 g) | | variable | | | | | | | |
| Canejuice | | | | | | 216.7 | g | | | | |
| (55% brix) | | | | | | | | | | | |
| Teknova CM-ura mix | One 1 L | | | | | | | | | | |
| | pack | | | | | | | | | | |
| Potassium phosphate | | | | 0.51 | g | 0-51 | g | | | | |
| monobasic | | | | | | | | | | | |
| Ammonium phosphate | | 13.8 | g | 0.87 | g | 0.87 | g | | | | |
| monobasic | | | | | | | | | | | |
| Ammonium phosphate | | 3.96 | g | 2.63 | g | 2.63 | g | | | | |
| dibasic | | | | | | | | | | | |
| Magnesium | | 0.493 | g | 0.55 | g | 0.55 | g | | | | |
| sulfate•7H₂O | | | | | | | | | | | |
| Yeast extract | | | | | | | | 10 | g | 10 | g |
| Peptone | | | | | | | | 20 | g | 20 | g |
| Thiamine HCl | | 200 | mcg | 0.1 | mg | 0.1 | mg | | | | |
| Niacin | | 3 | mg | 4 | mg | 4 | mg | | | | |
| Biotin | | 0.01 | mg | 0.01 | mg | 0.01 | mg | | | | |
| Calcium pantothenate | | 0.4 | mg | 0.4 | mg | 0.4 | mg | | | | |
| 1000 X trace | | 1 | ml | 1 | ml | 1 | ml | | | | |
| elements* | | | | | | | | | | | |
| pH (with ammonium | | 6.2 | | 6.8 | | 6.8 | | | | 6.0 | |
| hydroxide or | | | | | | | | | | | |
| phosphoric acid) | | | | | | | | | | | |
| MES ₍₂₋ | | | | | | | | | | 19.5 | g |
| _{₍}_{N-morpholino)} | | | | | | | | | | | |
| _{ethanesulfonic acid}_{₎} | | | | | | | | | | | |
| Betaine•H₂O | | | | 0.14 | g | 0.14 | g | | | | |
| Sodium Chloride | | 0.23 | g | | | | | | | | |
| Potassium Chloride | | 0.521 | g | | | | | | | | |

*1000 X Trace elements, per liter: 1.6 g FeCl₃•6H₂O, 0.1 g CuCl₂•2H₂O, 0.2 g ZnCl₂, 0.05 g H₃BO₃, 0.55 g MnCl₂•4H₂O, 10 mL 85% phosphoric acid.

TABLE 5
Results from pH controlled 7-liter fermentations of D-lactate producing
yeast strains with or without an integrated ZrFFZ1 cassette.

| | ZrFFZ1 | D-lactate | Final | Yield | Time | |
|---|---|---|---|---|---|---|
| Strain | cassette | Titer g/L | fructose g/L | g/g sucrose | Hours | Final pH |
| MYR2785 | No | 118 | 2.5 | 0.79 | 45 | 3.5 |
| SD1755 | Yes | 119 | 0 | 0.78 | 45 | 3.6 |
| MYR2879 | Yes | 117 | 0.4 | 0.79 | 48 | 3.5 |

TABLE 6
Results from pH controlled 5-liter fermentations of L-lactate producing
yeast strains with or without an integrated ZrFFZ1 cassette.

| | Experiment | ZrFFZ1 | L-lactate | Final | Yield | EOF |
|---|---|---|---|---|---|---|
| Strain | batch | cassette | Titer g/L | fructose g/L | g/g total sugar | (Hours) |
| MYR2893 | M30-M31 | No | 98.4 | 0 | 0.82 | 30 |
| JSS1397 | M32-M33 | Yes | 99.0 | 0 | 0.79 | 27 |

TABLE 7
Summary of strains derived from L-Lactate producing
yeasts and designed to constitutively overproduce
fructokinases from the KmPDC1 promoter

| Strain | Parental | Source of | Fructokinase gene | Site of |
|---|---|---|---|---|
| name | strain | fructokinase | installed | integration |
| MYR3049, | JSS1398 | K. marxianus | KmGLK1 | ADH1 |
| MYR3050 | | | | |
| MYR3051, | KMS1019 | K. marxianus | KmGLK1 | ADH1 |
| MYR3052 | | | | |
| MYR3053 | JSS1408 | K. marxianus | KmGLK1 | ADH1 |
| MYR3054, | JSS1398 | K. marxianus | KmRAG5 | SOR1 |
| MYR3055 | | | | |
| MYR3056, | KMS1019 | K. marxianus | KmRAG5 | SOR1 |
| MYR3057 | | | | |
| MYR3058, | JSS1408 | K. marxianus | KmRAG5 | SOR1 |
| MYR3059 | | | | |
| MYR3060, | JSS1398 | Tomato | SlFRK1 | AMD2 |
| MYR3061 | | | | |
| MYR3062, | KMS1019 | Tomato | SlFRK1 | AMD2 |
| MYR3063 | | | | |
| MYR3064, | JSS1408 | Tomato | SlFRK1 | AMD2 |
| M YR3065 | | | | |
| MYR3066, | JSS1398 | Thale cress | AtFRK1 | AMD2 |
| MYR3067 | | | | |
| MYR3068, | KMS1019 | Thale cress | AtFRK1 | AMD2 |
| MYR3069 | | | | |
| MYR3070, | JSS1408 | Thale cress | AtFRK1 | AMD2 |
| MYR3071 | | | | |
| MYR3072, | JSS1398 | Tomato | SlFRK2 | AMD4 |
| MYR3073 | | | | |
| MYR3074, | KMS1019 | Tomato | SlFRK2 | AMD4 |
| MYR3075 | | | | |
| MYR3076, | JSS1408 | Tomato | SlFRK2 | AMD4 |
| MYR3077 | | | | |
| MYR3078, | JSS1398 | Thale cress | AtFRK2 | AMD4 |
| MYR3079 | | | | |
| MYR3080, | KMS1019 | Thale cress | AtFRK2 | AMD4 |
| MYR3081 | | | | |
| MYR3082, | JSS1408 | Thale cress | AtFRK2 | AMD4 |
| MYR3083 | | | | |

### REFERENCES

[1] Abdel-Banat, B. M., Nonklang, S., Hoshida, H., and Akada, R. (2010) Random and targeted gene integrations through the control of non-homologous end joining in the yeast Kluyveromyces marxianus, Yeast 27, 29-39.
[2] Altschul, S. F., Gish, W., Miller, W., Myers, E. W., and Lipman, D. J. (1990) Basic local alignment search tool, J Mol Bioal 215, 403-410.
[3] Altschul, S. F., Madden, T. L., Schaffer, A. A., Zhang, J., Zhang, Z., Miller, W., and Lipman, D. J. (1997) Gapped BLAST and PSI-BLAST: a new generation of protein database search programs, Nucleic Acids Res 25, 3389-3402.
[4] Gietz, R. D. (2014) Yeast transformation by the LiAc/SS carrier DNA-PEG method, Methods Mol Biol 1205, 1-12.
[15] Goncalves, C., Wisecaver, J. H., Kominek, J., Oom, M. S., Leandro, M. J., Shen, X. X., Opulente, D. A., Zhou, X., Peris, D., Kurtzman, C. P., Hittinger, C. T., Rokas, A., and Goncalves, P. (2018) Evidence for loss and reacquisition of alcoholic fermentation in a fructophilic yeast lineage, Elife 7.
[6] Granot, D., Kelly, G., Stein, O., and David-Schwartz, R. (2014) Substantial roles of hexokinase and fructokinase in the effects of sugars on plant physiology and development, J Exp Bot 65, 809-819.
[7] Leandro, M. J., Cabral, S., Prista, C., Loureiro-Dias, M. C., and Sychrova, H. (2014) The high-capacity specific fructose facilitator ZrFfz1 is essential for the fructophilic behavior of Zygosaccharomyces rouxii CBS 732T, Eukaryot Cell 13, 1371-1379.
[8] Lobo, Z., and Maitra, P. K. (1982) Genetic evidence for distinct catalytic and regulatory subunits in yeast phosphofructokinase, FEBS Lett 139, 93-96.
[9] Michelson, T., et al. (2006) L(+)-Lactic acid producer Bacillus coagulans SIM-7 DSM 14043 and its comparison with Lactobacillus delbrueckii ssp. lactis DSM 20073, Enzyme and Microbial Technology 36, 861-867.
[10] Pina, C., Goncalves, P., Prista, C., and Loureiro-Dias, M. C. (2004) Ffz1, a new transporter specific for fructose from Zygosaccharomyces bailii, Microbiology 150, 2429-2433.
[11] Poudel, P., Tashiro, Y., and Sakai, K. (2016) New application of Bacillus strains for optically pure L-lactic acid production: general overview and future prospects, Biosci Biotechnol Biochem 80, 642-654.
[12] Rodicio, R., Strauss, A., and Heinisch, J. J. (2000) Single point mutations in either gene encoding the subunits of the heterooctameric yeast phosphofructokinase abolish allosteric inhibition by ATP, J Biol Chem 275, 40952-40960.
[13] Rudolph, H., Koenig-Rauseo, I., and Hinnen, A. (1985) One-step gene replacement in yeast by cotransformation, Gene 36, 87-95.
[14] Stein, O., and Granot, D. (2018) Plant Fructokinases: Evolutionary, Developmental, and Metabolic Aspects in Sink Tissues, Front Plant Sci 9, 339.
[15] Zhou, Y., Zhu, Y., Men, Y., Dong, C., Sun, Y., and Zhang, J. (2017) Construction of engineered Saccharomyces cerevisiae strain to improve that whole-cell biocatalytic production of melibiose from raffinose, J Ind Microbiol Biotechnol 44, 489-501.

1-9. (canceled)

10. A genetically engineered Kluyveromyces sp. yeast strain that is capable of producing lactic acid from carbon source selected from glucose, fructose, sucrose or a mixture thereof, wherein the genetically engineered yeast comprises at least one heterologous DNA cassette that confers production of a protein functioning as a fructose importer.

11. The genetically engineered Kluyveromyces sp. yeast strain of claim 10, wherein said fructose importer is functioned by facilitated diffusion.

12. The genetically engineered Kluyveromyces sp. yeast strain of claim 10, wherein said fructose importer is encoded by an exogenous FFZ1 gene, or a functional homolog thereof.

13. The genetically engineered Kluyveromyces sp. yeast strain according to claim 10, wherein said genetically engineered yeast strain has an improvement of fructose utilization as compared to the parental.

14. The genetically engineered Kluyveromyces sp. yeast strain according to claim 10, wherein said genetically engineered yeast strain is capable of consuming all measurable glucose and fructose with fructose consumption rate of at least 1.25 g L⁻¹ hr⁻¹.

15. The genetically engineered Kluyveromyces sp. yeast strain according to claim 10, wherein said genetically engineered yeast strain further comprises a cassette that confers expression of gene encoding a fructokinase or a hexokinase.

16. The genetically engineered Kluyveromyces sp. yeast strain according to claim 10, wherein Kluyveromyces sp. yeast strain is selected from Kluyveromyces lactis, Kluyveromyces marxianus, Kluyveromyces thermotolerans, Kluyveromyces aestuarii, Kluyveromyces africanus, Kluyveromyces bacillisporus, Kluyveromyces blattae, Kluyveromyces dobzhanskii, Kluyveromyces hubeiensis, Kluyveromyces lodderae, Kluyveromyces nonfermentans, Kluyveromyces piceae, Kluyveromyces sinensis, Kluyveromyces, Kluyveromyces waltii, Kluyveromyces wickerhamii, and Kluyveromyces yarrowii.

17. A method for producing lactic acid comprising growing said genetically engineered Kluyveromyces sp. yeast strain according to claim 10 in a liquid medium.

18. A fermentation process for lactic acid production, comprising employing said genetically engineered Kluyveromyces sp. yeast strain according to claim 10.


SEQUENCE LISTING (7 sequences)

SEQ ID NO: 1 - DNA, 2660 bp, Artificial Sequence
  Cassette for deleting the NEJ1 gene from Kluyveromyces marxianus.
gtacttgctggcgtatatgtgatacacaaactaaacctatggggtcctttataccaattcgctaatcaagcaacgggacaagcaactatgactttgaaacagactgtccgtaatttggtgcttgatgaagaccctaagcctgtatccacagagaaatactctggagatttgaaggccaggaaagaagatccttctaaagagagtacccatttataaggagcaaatataaaacaacaaaatagcaatcgcaaccacatattagataggtctgtagactatatccaatgcaccaaaatatggcatcctatttcctgtgccttatcactacatgatcattcgtttagatcttttaactaatattgtatataaccattttttaaggctgtagttcttacaaacttaaccttaaaaaacttagcatgggaaccttacaaagactactacgctgaaagaaatagtaacctaagcgctagattcggatttccttgcaaaaaggatatgtatttctaaccttttcttctagctctccgatcaagctggacctaattcagagcacctttgtagccaagtgtgaaagaacttcgaagaagcctcagaatccacatctgcatcttccttacaaagcgcttgcacaatcttcgaccagaaggttacaaatgccttcaattggtgtacactaaccggtacttccaccaattcattaggcagttgtttccaatcattgtggctaataccagaatttctgaagaaatcagagaacaccgcaatataatgcttgtaaattacccttgcgactgctttgaagtctgtcatttctttacggctgtacgaagtgaacattaactcgtcaccgtatatgcgtatcatagtacgatcaatggcctccacaatgacgttgaaaaattcgtcgcaggcgctaacggcaagggcgtggaaatccgaactggatacgccattgtatgtaggctcgcgttcaattggtttagggacctggatattttcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttctaaagagcttctgaaagcctcaacgaaagaagaaggactaatagatacactgcaggacatatacacaatattatcagaagaatccaattctaagtttgagtcattgggaagcaagatgtttctatccatccaaagaaaagattcgtctgagattgtcttagaagtggaaacctccagtgttgatgagagtcgagaacaaacgctactacatgagtgtactggatatttgatgaaaaatgcggtggttggaaatcatattcgaacatatatttcccacctcgcattagaaaaagatagggcgatcgaatttttagggaatactgccaaagattacggagctagcaatacaatacttaaatgggctcctgtaaactctccaaactacaaatcattgatgaaatttgatccaagtttggcatgggcggagagttttccaaatggaagcgtcaatctagacataatagatcttaatgaattaaatgaaattacacacaagag

SEQ ID NO: 2 - DNA, 5363 bp, Artificial Sequence
  Cassette for integrating and expressing ZrFFZ1 at the KmADH2 locus.
atgtctattccaactactcaaaagggtgttatcttctacgaaaacggtggtcaattgtactacaaggacatcccagtcccaaagccaaagtctaacgaacttttgatcaacgttaagtactccggtgtctgccacaccgatttgcacgcctggaagggtgactggccattggacaccaagttgccattggtcggtggtcacgaaggtgccggtgtcgtcgtcgccatgggtgacaacgtcaagggctggaagatcggtgaccttgccggtatcaaatggttgaacggttcttgtatgaactgtgaagaatgtgaattgtccaacgaatccaactgtccagacgctgacttgtccggttacacccacgacggttctttccaacaatacgctaccgctgacgctgtccaagccgctcacatcccagctggtaccgacttggctcaagtcgccccaatcttgtgtgccggtgttaccgtctacaaggctttgaagaccgctgaataaagcgtgaataatgaatggccttgtattcgtttttttccgagagaaaattaacaagagcgaaaaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttgggttatggaattagtatgggtatgtgatatgtggtaatatgtgatattgggttattgtgatttggaatactgaatatcgaatatgggatatggaatatggctatggcatggtatggtatgggatgggagtattctattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaatggtcaaaatagacgccagtgcagatttggactggaataatttgcctccccaacagaggttgggtagaatcgttggtggaaaatatattgaaaccgtgcacatggatgagtcatcaccagaagccatggagcgtccaatgacaaaagtcgagtgtgatatcccattaacttatactgaatctgtggaaaaggacggcaaagaatgggtaatgttaacttttgcaccaggtgatccagaaaacccatttaattggagtcttagattgaaatggtttctaacgactttactgtgcctaatgacacttttcatcggtctagccaccacggcttatagttctggtatagacagaatggcaaaacactttaacagttcttcggaagttggtcagttgggtcttttcaccttcaatgaaacatgttccattgcacctttgtttctagcgccattctgtgaattggttggtagaaaggtgatttacaccggtggttatctttgcttctgtctttgtttcatcggtcttgcattaggtaaaaatatggccactatcatttgtttgcgtgcttgtttaggtttgtttggttgcattggtaccattcttgttggtggtactttcgacgatatgtttgttgcagatgaaagagctattcctatggctctgtttgcatatgttgctatcctgggtaccgtgggtgctccaatctatgcaggttttattgatcaagccattggctggcgttggattgaaggtatccaagggttatctaatgttccattgttgattatcatctttttatttttcaaagaaactcgtggtggtgtcaccttgcaaaagagggccaagtctctacgtaaagataccggtgatgagagatgggtgtccaaggaagaattggaggctcctggtcttaaggatgctctttacaactcctctgttaaggctattaagatgttaatctccgagcccgtggtgtttttctttggtctttggatcagtttcgcatggttcctaacctttttgtttttgtctgttatcggtattactttctcacactttaagcactggagtgaaggtgttgcaggtttaccttatatctcccttgttattggtgttaccattggattcttaaccaactttttgcaaattcgcaaatacgaaaatatgagaaagaagtcaaagtatcctttaccacctgaaaaccgtttatatggtgctatgactggtgccgtgtttttacctattggtcttatgatctactctttcactcaataccactggttgcactggatcggtcctaacatcggtttggcatgcatctctctcggtatcttcttcatcttcgaatcctgttattcctacacttcggattgttatggtcctgatgcttcttccgccattgcaggtcaaggtttcatgcgtaatactttaggtgccgtttcaccattgtttggtaagtacatgttcttaaacatgcacagtcaatgggccggcctgttgtgtagtctcgtggctttcggtttgacattgttgcccttcgtcctttacaaatttggtcctgcaatccgtgccagatctaagcgtgctattgtgtaccatggtgaagaagacgatgaagatgacgaaattgctgaacagcaaagtagctcaagtactggaggaggcagatctttgcaagatgatgctcacgttgaaaatgctaacaacaaccagttattcgaaaagaacgaaggttctcgtgaatctgattctccagagactgctagagagactgattccctatcaccttccgaaaaggtagataaccatgctggagtatctcgtagcgtggaagaacactcagataccgtttctgaaagtgaattacatggccatactggtcgtgcatgaaatatctatatcacaagatgaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttggatccacaggacgggtgtggtcgccatgatcgcgtagtcgatagtggctccgtccggcgtagaggatcctcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttggatccacaggacgggtgtggtcgccatgatcgcgtagtcgatagtggctcaacttagactaaggaggtttggggcgcgccatgaaggctggtgactgggtcgccatctccggtgctgctggtggtctaggttccttggccgtccaatacgccaaggccatgggtttcagagtcctaggtatcgatggtggtgaaggtaaggaagaattgttcaagagcttgggtggtgaagtcttcattgatttcaccaagtctaaggacattgtcggtgaagtcatcaaggctaccaacggtggtgctcacggtgtcatcaacgtctccgtctccgaaaaggccatcgaatcctccatcgaatactgtagatccaacggtaccgtcgttctagtcggtttgccaaaggacgccaagtgtaagtccgatgtcttcaaccaagtcgtcaagtccatccacattgttggttcttacgtcggtaacagagctgacaccagagaagctcttgacttcttctgcagaggtctagtccacgccccaatcaaggttgtcggtttgtccaccttgccagaaatttacgaaaagatgga

SEQ ID NO: 3 - DNA, 6228 bp, Artificial Sequence
  Cassette from pJSS89 for integrating and expressing ZrFFZ1 by insertion in the middle of the KmADH6 ORF.
cccggggatcctctagagtcgagcggccgccgaaagcattgcgcttgtacggtacactactgcatattcggacgtggaaacggcaagaaaaaaaagccaggaatgcaagtgcgggaataagctacgtaccgcacgctgtatacttgtgcactagatgtttcttgtgcgcttcctatttgtgcgtgaagacgtagagaagattatacaatacaggtctcgagcgtgcgccgcgaatccagtagattatgtcagaagagcaaatgggaagaagcaaaagttggtagggtagagattatagatgaggacgggttttgatatgagagattagtaattagtaatcacagagagagagagatggaatagaatgtcttagagacacacatgcacgtttcacaatgtgtaaaagtataaaaagaagggagcattgaatcttttcttgtcaattgtttaagctaatgtagttagtatgaatttaaagagcgtactacagtaagctgatagatagaaggtatatccatactttagccaaataaagcgtgaataatgaatggccttgtattcgtttttttccgagagaaaattaacaagagcgaaaaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttgggttatggaattagtatgggtatgtgatatgtggtaatatgtgatattgggttattgtgatttggaatactgaatatcgaatatggatatggaatatggctatggcatggtatggtatgggatgggagtattctattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaatggtcaaaatagacgccagtgcagatttggactggaataatttgcctccccaacagaggttgggtagaatcgttggtggaaaatatattgaaaccgtgcacatggatgagtcatcaccagaagccatggagcgtccaatgacaaaagtcgagtgtgatatcccattaacttatactgaatctgtggaaaaggacggcaaagaatgggtaatgttaacttttgcaccaggtgatccagaaaacccatttaattggagtcttagattgaaatggtttctaacgactttactgtgcctaatgacacttttcatcggtctagccaccacggcttatattctggtatagacagaatggcaaaacactttaacagttcttcggaagttggtcagttgggtcttttcaccttcaatgaaacatgttccattgcacctttgtttctagcgccattctgtgaattggttggtagaaaggtgatttacaccggtggttatctttgcttctgtctttgtttcatcggtcttgcattaggtaaaaatatggccactatcatttgtttgcgtgcttgtttaggtttgtttggttgcattggtaccattcttgttggtggtactttcgacgatatgtttgttgcagatgaaagagctattcctatggctctgtttgcatatgttgctatcctgggtaccgtgggtgctccaatctatgcaggttttattgatcaagccattggctggcgttggattgaaggtatccaagggttatctaatgttccattgttgattatcatctttttatttttcaaagaaactcgtggtggtgtcaccttgcaaaagagggccaagtctctacgtaaagataccggtgatgagagatgggtgtccaaggaagaattggaggctcctggtcttaaggatgctctttacaactcctctgttaaggctattaagatgttaatctccgagcccgtggtgtttttctttggtctttggatcagtttcgcatggttcctaacctttttgtttttgtctgttatcggtattactttctcacactttaagcactggagtgaaggtgttgcaggtttaccttatatctcccttgttattggtgttaccattggattcttaaccaactttttgcaaattcgcaaatacgaaaatatgagaaagaagtcaaagtatcctttaccacctgaaaaccgtttatatggtgctatgactggtgccgtgtttttacctattggtcttatgatctactctttcactcaataccactggttgcactggatcggtcctaacatcggtttggcatgcatctctctcggtatcttcttcatcttcgaatcctgttattcctacacttcggattgttatggtcctgatgcttcttccgccattgcaggtcaaggtttcatgcgtaatactttaggtgccgtttcaccattgtttggtaagtacatgttcttaaacatgcacagtcaatgggccggcctgttgtgtagtctcgtggctttcggtttgacattgttgcccttcgtcctttacaaatttggtcctgcaatccgtgccagatctaagcgtgctattgtgtaccatggtgaagaagacgatgaagatgacgaaattgctgaacagcaaagtagctcaagtactggaggaggcagatctttgcaagatgatgctcacgttgaaaatgctaacaacaaccagttattcgaaaagaacgaaggttctcgtgaatctgattctccagagactgctagagagactgattccctatcaccttccgaaaaggtagataaccatgctggagtatctcgtagcgtggaagaacactcagataccgtttctgaaagtgaattacatggccatactggtcgtgcatgaacgttgtcattttcatttcatttcatttcataacatttctgttttcatttgtctttttttctgttattataattaacaaacgagaatggatcttataaattcgtaacgatggccttcataccacaattccatctgaattactaacactattaactttatatttttctttctgtctaacgaacggtgtaaagcatatgataataaattaactatataggttttaaaaaaagtatagaacgaacttaaatgatatcaggataattgaagatgtttttttttactttttgtttgcttatatattacaaatataatgggaggggtacggagataaaactgtaagcgggaacaatggttgtaaacgtcaaacttcatgactccaacaagttagacttttgcgtagcgtctgcacctgaaaatccggggccgataccaacaagctaacactaacaaatactgtagagacacggtacctagcaagttcctagacacataaacagacaatctagtacacacggaagccagacaaccgctacaggcactacttacgttatctgcatttatcctaatgaggcaaaaatctagaaaccatggagactaccccgcgattaaaggcgattaaaagaaagtaccacaacgtagaacagagccgaacttaaagtggaacaggtgtctacgagctgtacttcccgagctggtttctccaaacagccaatgatcataccaagatctgatgtatggcaacgttgttcacgtttgacatacgcagtatggttgacaagaatcagggctctcaagctcagttgagcggtttccgggtcaaattgctatgaattcagtgatgagaattaatgcaactcccttctttgtgactagttcctacactcaattgaaacacggcatgagaattataaaactctaaagcaggtctaaacttatactttgcatgtcgttgcatttcccccgtcttctaaaaaacataaccctttcaattatacttatatataatataatatgaattcgtcattctatgcagcattattatacagatccagatacaaatacgaagccattttacaaaaaaaaaaaagattatactacaaaagttttgaagtagaatcgtaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgacgtgttggtgttggtgcgcaagtcagatcctgtttggaatgtagacgttgtaaggaggacaacgaaccatactgtccaaagttcgtcactacttactcgcaaccatacccagaagatggttacgtttcccaaggtggttacgcctctcatatcagattgcatgagcactttgccattccaatcccagaatctctagaagccgcttacactgctcctctactttgcggtggtggtactgtctactctccattgaagagatacggctgtggtccaggtaagaaggttggtatcgtcggtatcggtggtattggtcacatgggtatccttttggccaaggctatgggtgctgaagtttacgccatctccagatctcacgctaaagaagaagctgccaagaaattgggtgctgaccactacattgctaccaaggatgaaggctgggaattggaaaactttgacaaattggacttgatcgttctatgtgccagctccttgaccgagcggccgcgccgggtcacccggccagcgac

SEQ ID NO: 4 - DNA, 6094 bp, Artificial Sequence
  Cassette from pJSS90 for integrating and expressing ZrFFZ1 with simultaneous deletion of the KmADH6 ORF.
cccggggatcctctagagtcgagcggccgctttgatatgagagattagtaattagtaatcacagagagagagagatggaatagaatgtcttagagacacacatgcacgtttcacaatgtgtaaaagtataaaaagaagggagcattgaatcttttcttgtcaattgtttaagctaatgtagttagtatgaatttaaagagcgtactacagtaagctgatagatagaaggtatatccatactttagccaaatagtcaaataatgtcctacccagatagtttccaaggtttcgctgtcgttaatgaccacaagaactggttgaacccagaaaagatcgtttacccagccaagaagttcggtcctcaagacgttgatgtcgagatcgaagcttgcggtgtttgtggtagtgatgtccattgtgccaacggtaactggggtaatcaaaagttgccattagttgttggtcacgaagtcatcggtaaggttatcagagttggtgaagaatgtaccactggtatcaaggttggtgactaaagcgtgaataatgaatggccttgtattcgtttttttccgagagaaaattaacaagagcgaaaaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttgggttatggaattagtatgggtatgtgatatgtggtaatatgtgatattgggttattgtgatttggaatactgaatatcgaatatgggatatggaatatggctatggcatggtatggtatgggatgggagtattctattttattttattcggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaatggtcaaaatagacgccagtgcagatttggactggaataatttgcctccccaacagaggttgggtagaatcgttggtggaaaatatattgaaaccgtgcacatggatgagtcatcaccagaagccatggagcgtccaatgacaaaagtcgagtgtgatatcccattaacttatactgaatctgtggaaaaggacggcaaagaatgggtaatgttaacttttgcaccaggtgatccagaaaacccatttaattggagtcttagattgaaatggtttctaacgactttactgtgcctaatgacacttttcatcggtctagccaccacggcttatagttctggtatagacagaatggcaaaacactttaacagttcttcggaagttggtcagttgggtcttttcaccttcaatgaaacatgttccattgcacctttgtttctagcgccattctgtgaattggttggtagaaaggtgatttacaccggtggttatctttgcttctgtctttgtttcatcggtcttgcattaggtaaaaatatggccactatcatttgtttgcgtgcttgtttaggtttgtttggttgcattggtaccattcttgttggtggtactttcgacgatatgtttgttgcagatgaaagagctattcctatggctctgtttgcatatgttgctatcctgggtaccgtgggtgctccaatctatgcaggttttattgatcaagccattggctggcgttggattgaaggtatccaagggttatctaatgttccattgttgattatcatctttttatttttcaaagaaactcgtggtggtgtcaccttgcaaaagagggccaagtctctacgtaaagataccggtgatgagagatgggtgtccaaggaagaattggaggctcctggtcttaaggatgctctttacaactcctctgttaaggctattaagatgttaatctccgagcccgtggtgtttttctttggtctttggatcagtttcgcatggttcctaacctttttgtttttgtctgttatcggtattactttctcacactttaagcactggagtgaaggtgttgcaggtttaccttatatctcccttgttattggtgttaccattggattcttaaccaactttttgcaaattcgcaaatacgaaaatatgagaaagaagtcaaagtatcctttaccacctgaaaaccgtttatatggtgctatgactggtgccgtgtttttacctattggtcttatgatctactctttcactcaataccactggttgcactggatcggtcctaacatcggtttggcatgcatctctctcggtatcttcttcatcttcgaatcctgttattcctacacttcggattgttatggtcctgatgcttcttccgccattgcaggtcaaggtttcatgcgtaatactttaggtgccgtttcaccattgtttggtaagtacatgttcttaaacatgcacagtcaatgggccggcctgttgtgtagtctcgtggctttcggtttgacattgttgcccttcgtcctttacaaatttggtcctgcaatccgtgccagatctaagcgtgctattgtgtaccatggtgaagaagacgatgaagatgacgaaattgctgaacagcaaagtagctcaagtactggaggaggcagatctttgcaagatgatgctcacgttgaaaatgctaacaacaaccagttattcgaaaagaacgaaggttctcgtgaatctgattctccagagactgctagagagactgattccctatcaccttccgaaaaggtagataaccatgctggagtatctcgtagcgtggaagaacactcagataccgtttctgaaagtgaattacatggccatactggtcgtgcatgaacgttgtcattttcatttcatttcatttcataacatttctgttttcatttgtctttttttctgttattataattaacaaacgagaatggatcttataaattcgtaacgatggccttcataccacaattccatctgaattactaacactattaactttatatttttctttctgtctaacgaacggtgtaaagcatatgataataaattcgtccggcgtagaggatcctcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaacgttgtcattttcatttcatttcatttcataacatttctgttttcatttgtctttttttctgttattataattaacaaacgagaatggatcttataaattcgtaacgatggccttcataccacaattccatctgaattactaacactattaactttatatttttctttctgtctaacgaacggtgtaaagcatatgataataaattaactatataggttttaaaaaaagtatagaacgaacttaaatgatatcaggataattgaagatgtttttttttactttttgtttgcttatatattacaaatataatgggaggggtacggagataaaactgtaagcgggaacaatggttgtaaacgtcaaacttcatgactccaacaagttagacttttgcgtagcgtctgcacctgaaaatccggggccgataccaacaagctaacactaacaaatactgtagagacacggtacctagcaagttcctagacacataaacagacaatctagtacacacggaagccagacaaccgctacaggcactacttacgttatctgcatttatcctaatgaggcaaaaatctagaaaccatggagactaccccgcgattaaaggcgattaaaagaaagtaccacaacgtagaacagagccgaacttaaagtggaacaggtgtctacgagctgtacttcccgagctggtttctccaaacagccaatgatcataccaagatctgatgtatggcaacgttgttcacgtttgacatacgcagtatggttgacaagaatcagggctctcaagctcagttgagcggtttccgggtcaaattgctatgaattcagtgatgagaattaatgcaactcccttctttgtgactagttcctacactcaattgaaacacggcatcgtgttggtgttggtgcgcaagtcagatcctgtttggaatgtagacgttgtaaggaggacaacgaaccatactgtccaaagttcgtcactacttactcgcaaccatacccagaagatggttacgtttcccaaggtggttacgcctctcatatcagattgcatgagcactttgccattccaatcccagaatctctagaagccgcttacactgctcctctactttgcggtggtggtactgtctactctccattgaagagatacggctgtggtccaggtaagaaggttggtatcgtcggtatcggtggtattggtcacatgggtatccttttggccaaggctatgggtgctgaagtttacgccatctccagatctcacgctaaagaagaagctgccaagaaattgggtgctgaccactacattgctaccaaggatgaaggctgggaattggaaaactttgacaaattggacttgatcgttctatgtgccagctccttgaccgagcggccgcgccgggtcacccggccagcgac

SEQ ID NO: 5 - DNA, 6398 bp, Artificial Sequence
  Plasmid pMS155 containing the integration cassette for swapping in the PaldhL expression cassette to replace the EcldhA expression cassette at any of the three loci.
agcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttgggttatggaattagtatgggtatgtgatatgtggtaatatgtgatattgggttattgtgatttggaatactgaatatcgaatatgggatatggaatatggctatggcatggtatggtatgggatgggagtattctattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataccaagcacaactactagttttaaaggatccatcctcttaaacagtacaaatcgcaaagaaaagctccacacccaaaccaaataattgcaatgtctaatattcaaaatcatcaaaaagttgtcctcgtcggtgacggtgccgtaggttctagttacgcattcgcgatggcactacaaggaatcgctgaagaattcgtcattgtcgacgttgttaaggatcgtacagttggggacgcattggaccttgaagatgctactccattcacagctccaaagaacatctactctggtgaatactcagactgcaaggatgctgacttagttgttatcacagctggcgcaccacaaaagccaggtgaaacacgtcttgaccttgttaacaagaacttaaacatcctttcaacgattgttaaaccagttgttgattctggttttgatggtatcttccttgttgctgctaacccagttgatatccttacttacgcaacatggaaattctctggcttccctaaggaaaaagttatcggttcaggtatctcacttgacacagctcgtttgcgcgtagctcttggtaagaaattcaacgttagcccagaatctgtagatgcttacatcttaggtgaacatggtgacagtgaatttgctgcttactcatcagctacaatcggtacaaagccattgcttgaaatcgctaaagaagaaggcgtttcaactgacgaattggctgaaatcgaagacagcgtacgtaacgcagcttacgaaatcatcaacaagaagggtgctacattctacggtgttggtactgcattgatgcgcatttctaaagcaattcttcgcgacgaaaacgccgtattgcctgttggtgcatacatggatggcgaatatggtttgaacgacatttacattggtactcctgcagttatcaatggtcaaggtctaaaccgcgttatcgaagcaccacttagcgatgacgaaaagaagaagatgactgactcagcaactactttgaagaaggttcttactgacggtctaaacgctcttgctgaaaaacaagacaaataaggcgcgggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagccaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtcgactaagagttactcggaaagagcagctgctcatagaagtccagttgctgccaagcttttaaacttgatggaagagaagaagtcaaacttatgtgcttctcttgatgttcgtaaaacagcagagttgttaaaattagtcgaggttttgggtccatatatctgtctattgaagacacatgtagatatcttggaggatttcagctttgagaataccattgtgccgttgaagcaattagcagagaaacacaagtttttgatatttgaagacaggaagtttgccgacattgggaacactgttaaattacaatacacgtctggtgtataccgtatcgccgaatggtctgatatcaccaatgcacacggtgtgactggtgcgggcattgttgctggtttgaagcaaggtgccgaggaagttacgaaagaacctagagggttgttaatgcttgccgagttatcgtccaaggggtctctagcgcacggtgaatacactcgtgggaccgtggaaattgctaagagtgataaggactttgttattggatttattgctcaaaacgatatgggtggaagagaagagggctacgattggttgatcatgacgccaggtgttggtcttgatgacaaaggtgatgctttgggacaacaatacagaactgtggatgaagttgttgccggtggatcagacatcattattgttggtagaggtcttttcgcaaagggaagagatcctgtagtggaaggtgagagatacagaaaggcgggatgggacgcttacttgaagagagtaggcagatccgcttaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaagaactggtctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcagg

SEQ ID NO: 6 - DNA, 8515 bp, Artificial Sequence
  Plasmid pRY789 containing a ZrFFZ1 expression cassette for integration at the HO locus of Saccharomyces cerevisiae.
tgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgtcgcatgctcccggccgccatggccgcggacagcatcaaactgtaagattccgccacattttatacactctggtcctttaactggcaaaccttcgggcgtaatgcccaatttttcgcctttgtcttttgcctttttcacttcacgtgcttctggtacatacttgcaatttatacagtgatgaccgctgaatttgtatcttccatagcatctagcacatactcgatttttaccactccaatctttataaaaatacttgattccctttctgggacaagcaacacagtgttttagattctttttttgtgatattttaagctgttctcccacacagcagcctcgacatgatttcacttctattttgttgccaagcaagaaatttttatggccttctatcgtaagcccatatacagtactctcaccctggaaatcatccgtgaagctgaaatatacgggttccctttttataattggcggaacttctcttgttttgtgaccacttcgacaatatgacaaaacattctgtgaagttgttcccccagcaacattacagtcgtatgtaaattgacattggacttttcttccttcaatgatttcctccctagctgacctggtcgtcacagtagctgacataccaagagatcttgaaatatggacaattccgtccataatggatgaataaacagtttgaattgctattttataagattcagggccttcgccctttttcacaacgtacccatctgagtcgatcaagccggctaagaatgcttcacgaacttctatatgctcgccgtacataaattcagggatttgcttctctccatcaagatcccttttaaactttaaaattgtgacagctttccagaatggattatttttcctcaaattccttgtcttcctgttttcatctggaccatctccataatgaagccttacatgtttggcacgtagcggaacgtgatcgtcacaaaccgtaaggtagagaccccagattttcgcattttctcttaaactctccattagcttaggatccaagctatctactgagatttctggctcttttgttgtactgtcacctaaccacagacccagcgtgaataatgaatggccttgtattcgtttttttccgagagaaaattaacaagagcgaaaaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttgggttatggaattagtatgggtatgtgatatgtggtaatatgtgatattgggttattgtgatttggaatactgaatatcgaatatgggatatggaatatggctatggcatggtatggtatgggatgggagtattctattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaatggtcaaaatagacgccagtgcagatttggactggaataatttgcctccccaacagaggttgggtagaatcgttggtggaaaatatattgaaaccgtgcacatggatgagtcatcaccagaagccatggagcgtccaatgacaaaagtcgagtgtgatatcccattaacttatactgaatctgtggaaaaggacggcaaagaatgggtaatgttaacttttgcaccaggtgatccagaaaacccatttaattggagtcttagattgaaatggtttctaacgactttactgtgcctaatgacacttttcatcggtctagccaccacggcttatagttctggtatagacagaatggcaaaacactttaacagttcttcggaagttggtcagttgggtcttttcaccttcaatgaaacatgttccattgcacctttgtttctagcgccattctgtgaattggttggtagaaaggtgatttacaccggtggttatctttgcttctgtctttgtttcatcggtcttgcattaggtaaaaatatggccactatcatttgtttgcgtgcttgtttaggtttgtttggttgcattggtaccattcttgttggtggtactttcgacgatatgtttgttgcagatgaaagagctattcctatggctctgtttgcatatgttgctatcctgggtaccgtgggtgctccaatctatgcaggttttattgatcaagccattggctggcgttggattgaaggtatccaagggttatctaatgttccattgttgattatcatctttttatttttcaaagaaactcgtggtggtgtcaccttgcaaaagagggccaagtctctacgtaaagataccggtgatgagagatgggtgtccaaggaagaattggaggctcctggtcttaaggatgctctttacaactcctctgttaaggctattaagatgttaatctccgagcccgtggtgtttttctttggtctttggatcagtttcgcatggttcctaacctttttgtttttgtctgttatcggtattactttctcacactttaagcactggagtgaaggtgttgcaggtttaccttatatctcccttgttattggtgttaccattggattcttaaccaactttttgcaaattcgcaaatacgaaaatatgagaaagaagtcaaagtatcctttaccacctgaaaaccgtttatatggtgctatgactggtgccgtgtttttacctattggtcttatgatctactctttcactcaataccactggttgcactggatcggtcctaacatcggtttggcatgcatctctctcggtatcttcttcatcttcgaatcctgttattcctacacttcggattgttatggtcctgatgcttcttccgccattgcaggtcaaggtttcatgcgtaatactttaggtgccgtttcaccattgtttggtaagtacatgttcttaaacatgcacagtcaatgggccggcctgttgtgtagtctcgtggctttcggtttgacattgttgcccttcgtcctttacaaatttggtcctgcaatccgtgccagatctaagcgtgctattgtgtaccatggtgaagaagacgatgaagatgacgaaattgctgaacagcaaagtagctcaagtactggaggaggcagatctttgcaagatgatgctcacgttgaaaatgctaacaacaaccagttattcgaaaagaacgaaggttctcgtgaatctgattctccagagactgctagagagactgattccctatcaccttccgaaaaggtagataaccatgctggagtatctcgtagcgtggaagaacactcagataccgtttctgaaagtgaattacatggccatactggtcgtgcatgaaatatctatatcacaagatggactcaatgttgagcaaagcaggacgagaaaaaaaaaaataatgattgttaagaagttcatgaaaaaaaaaagaaaaaatactcaaatacttataacagagtgattaaataataaacgacagtataccctatcaggtattcagatagttttatttttgtaggtatataatctgaagcctttgaactattttctcgtatatatcatggagtatacattgcattagcaacattacatactagtgacttttgagaagtggcttttggtttctcaattttttgaaggaaacatccttggacctgtggcaactcctccaataattcttcgaagttatgttgaacttttcgtggtttcacacctaataacgcccagcggtagtacaattcaaagtagtaggtaccaatggtagtactagtgggtgatagtagcaaatttctctatgaaaatgaattgttgctctgcaatgatgttaatgtggacatgacatgaattttcgcgatatgttgatgtaaataataacatgattttcgtggatcatttcgtaaaccataggtttatttcgtgaattactaagagaaaggcatagaaacaggacttgcgaaccctttctgtaatatgtcataatatttcaatacacgcacaaaaaaggtacgttaatttccatggtaatcgtgtatttagttacatcacttttcgtgacacacatgaatacatttgcccttaagccctgtgtaggattgatttcttttttcgtgacgcacatgtcttcaactgcatcaatttccatgagacacacacgtctaccatgttgtcagatctcatgaatctttcatgtattcattcacatcattttcgtggatcctctaattgtatcgagatcacttttcgtgatccgctaatcagcgacggtcacattaggtttgccaagtcagggtatgaaccatacgatcagttttcgtgaacctggtacgtatattgtggcgtttgtgtatattttcattctttgacaacaatcaataccaacctcaaataggaaaagtaataagtttggcgttacaccccaaaagacgccaaacggatcgaacttactcaatagcaattagcgagacaaaacctacgttaagacctgtaaccgatttatcaaagcactctgcggttctttcttgggaatattacctggacattttgtgccctcaagaaacgaggctctacgagcctgttggagcccctcagacattagccgccacgaatcaaactttttacgcgattcggcccaaatcagtttctcacagatcattcgtagagtgaaaaagcacatcgattatttgatacccctttgggttaattactgttgaggtcttttctatttctgattgaaatttttgctccagcattatagcatgctggttttttttttaaattgatgtatctcatcgcaggcacgggcagtacagtgccctgagcgtagggaaaaatgaaaaaaaggatgtaacttttaacataattccagcacgcagcgattgggtataatgaagattgttaagttcaacaacatgtgaaattcttagctccgcaacttgcttgaattatgagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaa

SEQ ID NO: 7 - DNA, 8458 bp, Artificial Sequence
  Plasmid pBc-ldhL-int containing the integration cassette for swapping in the BcldhL expression cassette to replace the EcldhA expression cassette at any of the three loci.
atgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggcagcgtgaataatgaatggccttgtattcgtttttttccgagagaaaattaacaagagcgaaaaaaaaaacgggcttcggtgaaaatcgggtgaatatgcaactagcgggacgaatgctctggaaatgcatatcctatgcaactagcgggatgaacaaatctcaccccagaattcgcaggaaaaaacaggaaaaaaaaaaagaaggccaccacggccacaaagaccacaaagaccacaaaaaaaaacaaaaaacaaccgtcccagcttccagtgtttggaatactggaacacaggaagccgcataagagtgggcgttgcacaggaagccaggcccagaagccccagagttacttttttttttttgttttttccttctgttcgctgtgcccgcatcagatgatgcgcctttatttacgatgccaatgcgaatagcaccagtgagagcaccagtaaaagcatacgcatacacatacacacatagagcaagcaagcaggctagcaaccaggaaaggctgccagtgactgctactgggtgtctaagaaccgtagggcggattattgttgcggtggttggttgcgggtggttatgcgatggtacggtgcagaatcgtacggtgttgggttatggaattagtatgggtatgtgatatgtggtaatatgtgatattgggttattgtgatttggaatactgaatatcgaatatgggatatggaatatggctatggcatggtatggtatgggatgggagtattctattttattttattctggttcctgcgtttagggtagggtaggaagaaggtgagtgcttttgtatataagtggagtgtctggatcagttttgtggattgtgaatgttagtttcccctttaatgtatatttgtattatttgcttttgagtactcaataaccaagcacaactactagttttaaaggatccatcctcttaaacagtacaaatcgcaaagaaaagctccacacccaaaccaaataattgcaatgaagaaggtgaatcgtattgccgtggtcggcactggcgcagttggtacgagttactgctacgctatgataaatcagggggttgccgaggagttggtgttgatagatattaatgaggctaaggccgaaggggaagcaatggatttgaatcacggtcttccatttgctccgactcccaccagagtttggaagggcgactacagtgactgtggtaccgcagacctagtagtaatcaccgccgggtccccccagaagcccggagaaacccgtcttgacttggtttctaagaacgcaaaaatctttaaaggcatgattaaatcaataatggacagtggtttcaacggcattttcttggtagcatcaaaccccgttgatattcttacatacgttacgtggaaagaatcagggttgccgaaggagcacgtgataggttcaggaacagttcttgactcagcccgtctaagaaactcactttccgcccagtttggaattgacccaagaaatgtccacgctgccattataggtgaacacggggacacggagttacccgtctggtctcatactaacattggctacgacacgattgagtcgtacttacaaaaaggtatcatagacgaaaagacgcttgatgatatctttgtcaatactagagacgctgcataccacatcatcgaaaggaagggagccacattctatggaataggcatgtcgttgactaggatcaccagggccattttgaacaatgagaatagtgttttaacggttagtgccttcttagagggacaatatggtaattccgacgtatatgtcggagtaccggcaattattaataggcaaggtatacgtgaagtcgtagagataaaacttaatgagaaggagcaagaacagtttaatcacagtgttaaggttttaaaggagaccatggcccccattttgtaagagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgacagtccaaatgttgatttatcccagtccgattcatcagggttgtgaagcattttgtcaatggtcgaaatcacatcagtaatagtgcctcttacttgcctcatagaatttctttctcttaacgtcaccgtttggtcttttatagtttcgaaatctatggtgataccaaatggtgttcccaattcatcgttacgggcgtattttttaccaattgaagtattggaatcgtcaattttaaagtatatctctcttttacgtaaagcctgcgagatcctcttaagtatagcggggaagccatcgttattcgatattgtcgtaacaaatactttgatcggcgctatctgtaatggaaaccaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatgtcgactaagagttactcggaaagagcagctgctcatagaagtccagttgctgccaagcttttaaacttgatggaagagaagaagtcaaacttatgtgcttctcttgatgttcgtaaaacagcagagttgttaaaattagtcgaggttttgggtccatatatctgtctattgaagacacatgtagatatcttggaggatttcagctttgagaataccattgtgccgttgaagcaattagcagagaaacacaagtttttgatatttgaagacaggaagtttgccgacattgggaacactgttaaattacaatacacgtctggtgtataccgtatcgccgaatggtctgatatcaccaatgcacacggtgtgactggtgcgggcattgttgctggtttgaagcaaggtgccgaggaagttacgaaagaacctagagggttgttaatgcttgccgagttatcgtccaaggggtctctagcgcacggtgaatacactcgtgggaccgtggaaattgctaagagtgataaggactttgttattggatttattgctcaaaacgatatgggtggaagagaagagggctacgattggttgatcatgacgccaggtgttggtcttgatgacaaaggtgatgctttgggacaacaatacagaactgtggatgaagttgttgccggtggatcagacatcattattgttggtagaggtcttttcgcaaagggaagagatcctgtagtggaaggtgagagatacagaaaggcgggatgggacgcttacttgaagagagtaggcagatccgcttaatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggttggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattcgttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgt